(19) United States
(12) Patent Application Publication
(10) Pub. No.: US 2008/0311613 A1
(43) Pub. Date: Dec. 18, 2008

(54) ARTIFICIAL SKIN SURFACE FILM LIQUIDS

(75) Inventors: Aleksandr B. Stefaniak, Morgantown, WV (US); Christopher J. Harvey, Rillton, PA (US)

Correspondence Address: KLARQUIST SPARKMAN, LLP, 121 S.W. SALMON STREET, SUITE 1600, PORTLAND, OR, 97204, US

(73) Assignee: The Government of the U.S.A. as represented by the Secretary of the Dept. of Health & Human Services

(21) Appl. No.: 12/137,391

(22) Filed: Jun. 11, 2008

Related U.S. Application Data
(60) Provisional application No. 60/934,861, filed on Jun. 14, 2007.

(51) Int. Cl. C12Q 1/02

(52) U.S. Cl. 435/29

(57) ABSTRACT

An artificial skin surface film liquid (SSFL) is disclosed that mimics the chemical composition and biological action of both human sweat and sebum. The artificial sweat formulation contains a comprehensive combination of constituents. When combined with the sebum component it is particularly effective at modeling the effect of the skin surface film liquid on agents in contact with the skin. Also provided are methods of making and using the disclosed artificial skin surface compositions.

### CROSS-REFERENCE TO RELATED APPLICATION

This application claims the benefit of priority under 35 U.S.C. §119 to U.S. Provisional Application No. 60/934,861 filed on Jun. 14, 2007, which is incorporated herein by reference in its entirety

### FIELD

The present disclosure relates to artificial skin surface compositions that mimic the properties of human sweat and sebum on the skin and methods of making and using compositions thereof.

### BACKGROUND

The skin is the largest human organ, and its interaction with the environment has many important health consequences. For example, chemicals may be unintentionally leached from jewelry, textiles or cosmetics to cause contact dermatitis, skin irritation or toxicity. Drugs that are applied in topical compositions to the skin can interact with the skin surface in a way that affects the bioavailability of the active pharmaceutical agent. The skin surface film liquid (SSFL) is a significant factor that affects the interaction of the skin with such agents.

Sweat is secreted by sweat glands, which are tubular structures having a coil portion and a duct portion. The coil portion is located in the dermis of the skin and secretes a precursor sweat solution. The duct portion in the epidermis of the skin modifies the precursor sweat solution by reabsorbing ionic constituents (mostly sodium and chloride) before the solution reaches the skin surface where it is secreted as sweat. Sweat on the skin surface is the modified product of an active secretory function of epithelial cells that line the coiled portion of the sweat gland and constituents formed as by-products of the skin surface maturation and desquamation processes and metabolism by skin bacteria.

Sebum is secreted by pilosebaceous units, which are sebaceous glands physically connected via a sebaceous duct to a hair follicle. Sebaceous glands are present in all areas of the skin except for the palms of the hands and soles of the feet. Undifferentiated sebocyte cells, located at the periphery of a sebaceous gland, are pushed through a maturation zone in the gland, where they are filled with a freshly synthesized lipid mixture to develop into differentiated sebocyte cells. These cells then pass through a zone of necrosis in the gland where they increase 100- to 150-fold in volume by accumulating more lipids to form mature sebocyte cells. This lipid accumulation causes the mature sebocyte cells to disintegrate and release sebum into the sebaceous duct. Sebum is then secreted via hair follicles onto the surface of the skin where it mixes with epidermally derived lipids.

Human SSFL is a mixture of approximately 50% sweat and 50% sebum. The composition of an artificial SSFL is important for accurate in vitro modeling of potential chemical release on human skin. To provide meaningful results about potential chemical release and percutaneous absorption of agents in contact with the skin, a comprehensive artificial SSFL is needed that accurately mimics human SSFL. Unfortunately, a comprehensive understanding of the chemical composition of SSFL has not previously been available.

Prior SSFLs have primarily attempted to mimic the chemical content of sweat by including in it such components as sodium, chloride, potassium, lactic acid, amino acids and urea. One particularly comprehensive synthetic sweat composition was disclosed by Boman et al. (Contact Dermatitis 9: 159-160, 1983) which incorporated 14 constituents of sweat maintained at a pH of 5.1. This composition included Na⁺, Cl⁻, OH⁻, K₂SO₄, KCl, Ca(H₂PO₄)₂.H₂O, urea, arginine, histidine, valine, leucine, threonine, glucose, NH₃ and lactic acid.

Another artificial sweat formulation, the most commonly used today, is defined by the European Committee for Standardization (CEN) in EN 1811 as a simple solution of 0.5% NaCl, 0.1% urea and 0.1% lactic acid, with a pH adjusted to 6.5 using NH₄OH. The ISO standard ISO 3160-2 includes 20 g/L NaCl, 17.5 g/L NH₄OH, 5 g/L acetic acid and 15 g/L d,l-lactic acid with the pH adjusted to 4.7 by NaOH.

One of the more comprehensive summaries of the composition of eccrine sweat and the aqueous film surface was provided in Rothman et al., Physiology and Biochemistry of the Skin, University of Chicago Press (1954), chapter 7. This reference noted that eccrine sweat contains nitrogenous compounds, glucose, lactic acid, water-soluble vitamins and electrolytes. However, even this detailed treatment failed to disclose a composition that closely mimicked the complex content of SSFL.

The chemical composition of an artificial SSFL is important for accurate in vitro modeling of potential dissolution on human skin. For example, gold readily dissolves in artificial SSFL that contains certain sulfur-containing amino acids (Brown et al., Inorganica Chimica Acta 67: 27-30, 1982; Rapson, Gold Bull 15: 19-20, 1982; and Rapson, Contact Dermatitis 13: 56-65, 1985), but not in artificial SSFL that lacks these constituents (Lidén et al., Contact Dermatitis 39: 281-285, 1998a). Copper dissolution decreases as the sodium chloride concentration of artificial SSFL increases (Boman et al., Contact Dermatitis 9: 159-160, 1983). Additionally, the amount of dissolution from a test article in an individual constituent of artificial SSFL can be less than the amount of dissolution in the SSFL mixture (Collins, Br. J. Industrial Med. 14(3): 191-197, 1957; Hemingway and Molokhia, Contact Dermatitis 16: 99-105, 1987; Stauber et al., The Science of the Total Environment 74: 235-247, 1994).

The role of individual artificial SSFL constituents in the dissolution of metals, coupled with the observation that dissolution levels in individual constituents can differ from that of the mixture, highlight the need for a comprehensive artificial SSFL and in vitro test system that accurately match SSFL in vivo. A better understanding of material bioavailability will also improve the quality of data available for risk decision making to protect even the most susceptible persons from adverse health effects due to chemicals that leach from articles in contact with the skin.

### SUMMARY

The inventors have found that prior sweat formulations are unable to mimic the chemical complexity of SSFL, and therefore are unable to accurately model the interaction of environmental agents (such as consumer products and drugs) with the skin. For example, the inventors identified and critically reviewed 45 prior formulations of artificial sweat and 18 formulations of artificial sebum (in studies published from 1940 through 2005). This review determined that human sweat has a median pH of 5.3 and contains at least 60 different chemical constituents (see Table 1). Among the 45 formulations of artificial sweat, most lacked many of the constituents in human sweat. Additionally, concentrations of constituents in these artificial sweat formulations were often not within ranges found in human sweat.

Disclosed herein are artificial SSFL compositions that mimic the properties of human sweat and sebum on the skin. In some disclosed embodiments, the artificial SSFL composition includes an artificial sweat composition that includes electrolytes, ionic constituents, organic acids, carbohydrates, nitrogenous substances and vitamins in concentrations found in human sweat, and maintained at a pH of 4.8-5.8, for example 5.0-5.5.

In a particular example, the electrolytes include sodium, chloride, calcium, potassium, magnesium, phosphate and bicarbonate; the ionic constituents include sulfate, sulfur, fluoride, phosphorous, bromine, cadmium, copper, iodide, iron, lead, manganese, nickel, zinc; the organic acids include lactic acid, pyruvic acid, butyric acid, acetic acid, hexanoic acid, propionic acid, isobutyric acid, and isovaleric acid; the carbohydrates include glucose; the amino acids include alanine, arginine, aspartic acid, citrulline, glutamic acid, glycine, histidine, isoleucine, leucine, lysine, ornithine, phenylalanine, threonine, tyrosine, and valine; the nitrogenous substances include ammonium, uric acid, creatinine and creatine; and the vitamins include thiamine, riboflavin, nicotinic acid, pantothenic acid, pyridoxine, folic acid, ascorbic acid, dehydroascorbic acid, inositol, choline and p-aminobenzoic acid, and the artificial sweat composition is maintained at a pH of 5.1-5.5.

In a more particular example, the electrolytes are present in a molar concentration of Na (3.0-3.5×10⁻²), Cl (2.0-2.5×10⁻²), Ca (5.0-5.5×10⁻³), K (5.8-6.2×10⁻³), Mg (1.0-1.5×10⁻⁴), PO₄ (2.8-3.4×10⁻⁴), and HCO₃ (2.7-3.3×10⁻³); the ionic constituents are present in a concentration of SO₄ (3.9-4.5×10⁻⁴), S (2.0-2.6×10⁻³), F (0.8-1.4×10⁻⁵), P (1.0-1.6×10⁻⁵), Br (2.0-2.6×10⁻⁶), Cd (1.5-2.1×10⁻⁸), Cu (9.1-9.7×10⁻⁷), I (6.8-7.4×10⁻⁸), Fe (9.5-10.1×10⁻⁶), Pb (0.9-1.5×10⁻⁷), Mn (0.8-1.4×10⁻⁶), Ni (3.9-4.5×10⁻⁷), and Zn (1.0-1.6×10⁻⁵); the organic acids and carbohydrates are present in concentrations of lactic acid (1.1-1.7×10⁻²), pyruvic acid (1.5-2.1×10⁻⁴), butyric acid (2.1-2.7×10⁻⁶), acetic acid (1.0-1.6×10⁻⁴), hexanoic acid (8.7-9.3×10⁻⁷), propionic acid (3.2-3.8×10⁻⁶), isobutyric acid (7.7-8.3×10⁻⁷), isovaleric acid (0.8-1.4×10⁻⁶), and glucose (1.4-2.0×10⁻⁴); the amino acids are present in concentrations of alanine (3.3-3.9×10⁻⁴), arginine (7.5-8.1×10⁻⁴), aspartic acid (3.1-3.7×10⁻⁴), citrulline (3.7-4.3×10⁻⁴), glutamic acid (3.4-4.0×10⁻⁴), glycine (3.6-4.2×10⁻⁴), histidine (4.9-5.5×10⁻⁴), isoleucine (1.4-2.0×10⁻⁴), leucine (1.8-2.4×10⁻⁴), lysine (1.2-1.8×10⁻⁴), ornithine (1.2-1.8×10⁻⁴), phenylalanine (1.0-1.6×10⁻⁴), threonine (4.2-4.8×10⁻⁴), tryptophan (5.2-5.8×10⁻⁵), tyrosine (1.4-2.0×10⁻⁴), and valine (2.2-2.8×10⁻⁴); the nitrogenous substances are present in a concentration of NH₃ (4.9-5.5×10⁻³), urea (0.7-1.3×10⁻²), uric acid (5.6-6.2×10⁻⁵), creatinine (8.1-8.7×10⁻⁵), and creatine (1.2-1.8×10⁻⁵); the vitamins are present in a concentration of thiamine (4.7-5.3×10⁻³), riboflavin (1.7-2.5×10⁻³), niacin (3.8-4.4×10⁻¹), pantothenic acid (1.0-1.6×10⁻¹), pyridoxine (0.7-1.3×10⁻⁸), folic acid (1.3-1.9×10⁻⁸), ascorbic acid (0.7-1.3×10⁻⁵) and/or its oxidation product dehydroascorbic acid (0.8-1.4×10⁻⁵), inositol (1.3-1.9×10⁻⁶), choline (2.3-2.9×10⁻⁵), and p-aminobenzoic acid (6.8-7.4×10⁻⁸).

In an even more particular example, artificial SSFL constituent concentrations match those of human sweat. The electrolytes are present in molar concentrations of Na (3.1×10⁻²), Cl (2.3×10⁻²), Ca (5.2×10⁻³), K (6.1×10⁻³), Mg (8.2×10⁻⁵), PO₄ (3.1×10⁻⁴), and HCO₃ (3×10⁻³); ionic constituents are present in a concentration of SO₄ (4.2×10⁻⁴), S (2.3×10⁻³), F (1.1×10⁻⁵), P (1.3×10⁻⁵), Br (2.3×10⁻⁶), Cd (1.8×10⁻⁸), Cu (9.4×10⁻⁷), I (7.1×10⁻⁸), Fe (9.8×10⁻⁶), Pb (1.2×10⁻⁷), Mn (1.1×10⁻⁶), Ni (4.2×10⁻⁷), and Zn (1.3×10⁻⁵); organic acids and carbohydrates are present in concentrations of lactic acid (1.4×10⁻²), pyruvic acid (1.8×10⁻⁴), butyric acid (2.4×10⁻⁶), acetic acid (1.3×10⁻⁴), hexanoic acid (9.0×10⁻⁷), propionic acid (3.5×10⁻⁶), isobutyric acid (8.0×10⁻⁷), isovaleric acid (1.1×10⁻⁶), and glucose (1.7×10⁻⁴); amino acids are present in concentrations of alanine (3.6×10⁻⁴), arginine (7.8×10⁻⁴), aspartic acid (3.4×10⁻⁴), citrulline (4.0×10⁻⁴), glutamic acid (3.7×10⁻⁴), glycine (3.9×10⁻⁴), histidine (5.2×10⁻⁴), isoleucine (1.7×10⁻⁴), leucine (2.1×10⁻⁴), lysine (1.5×10⁻⁴), ornithine (1.5×10⁻⁴), phenylalanine (1.3×10⁻⁴), threonine (4.5×10⁻⁴), tryptophan (5.5×10⁻⁵), tyrosine (1.7×10⁻⁴), and valine (2.5×10⁻⁴); the nitrogenous substances are present in concentrations of NH₃ (5.2×10⁻³), urea (1.0×10⁻²), uric acid (5.9×10⁻⁵), creatinine (8.4×10⁻⁵), and creatine (1.5×10⁻⁵); the vitamins are present in concentrations of thiamine (5.0×10⁻³), riboflavin (2.0×10⁻³), niacin (4.1×10⁻¹), pantothenic acid (1.3×10⁻¹), pyridoxine (1.0×10⁻⁸), folic acid (1.6×10⁻⁸), ascorbic acid (1.0×10⁻⁵) and/or its oxidation product dehydroascorbic acid (1.1×10⁻⁵), inositol (1.6×10⁻⁶), choline (2.6×10⁻⁵), and p-aminobenzoic acid (7.1×10⁻⁸); and the composition is at a pH of about 5.3.

The composition can, in some examples, further include a sebum formulation that includes squalene, wax esters, triglycerides, free fatty acids, cholesterol esters, free cholesterol and vitamin E in concentrations found in human sebum. For example, the sebum formulation may include 8-12% squalene, 23-27% wax esters, 30-35% triglycerides, 25-30% free fatty acids, 1-3% cholesterol esters, and 3-5% free cholesterol, and in more particular examples 10% squalene, 25% wax esters, 33% triglycerides, 28% free fatty acids, 2% cholesterol esters, and 4% free cholesterol. The wax esters may include palmityl palmitate and oleyl oleate; the triglycerides include tristearin and triolein; the free fatty acids include stearic, palmitic and oleic acid; the cholesterol esters include cholesteryl oleate; and the Vitamin E is (±)-α-tocopherol.

More detailed formulations of artificial sweat and sebum formulations are described below, such as in the Example Section. The artificial sweat and sebum formulations may be prepared and stored separately, and combined at the time of dissolution testing. Methods are also disclosed for performing dissolution testing using the artificial sweat and sebum formulations. Although dissolution testing in the combined sweat and sebum formulations provide particularly good results, testing can also be performed separately with the sweat and sebum formulations to determine how these subcomponents of SSFL interact with particular test objects. Test methods include testing dissolution of an object (such as a metal, cloth or pharmaceutical object) in human sweat by exposing the object to the composition for a sufficient period of time for it to at least partially dissolve, then quantifying dissolution of the object in the composition. The comprehensive artificial sweat and sebum formulations disclosed herein have been found to provide greater and more accurate dissolution of objects than has been obtained with prior formulations.

Methods of making an artificial sweat composition are also disclosed, in which a volume of water of about 70-80% of a final desired liquid volume of the artificial sweat composition are provided. The primary electrolytes and ionic constituents are added to the water to form a primary solution, then the organic acids, amino acids, nitrogenous substances and vitamins are added to the primary solution. Solubility of vitamins in the artificial sweat is enhanced at a pH greater than 4.8, for example a pH of 4.8-5.8, for example 5.0-5.5 or 5.3. Methods are also disclosed for preserving the separately stored sebum formulation by refrigerating it in a container under a nitrogen gas blanket, for example at less than 10° C., such as at 4° C.

The foregoing and other features of the disclosure will become more apparent from the following detailed description, which proceeds with reference to the accompanying figures.

### BRIEF DESCRIPTION OF THE DRAWINGS

FIG. 1 is a bar graph illustrating similar dissolution rates of cobalt by three disclosed artificial SSFL formulations.

FIG. 2 is a bar graph illustrating enhanced dissolution of tungsten powder and cobalt powder by a disclosed artificial SSFL formulation as compared to a CEN 1811 sweat formulation. Dissolution rates of the poorly soluble tungsten carbide material were not observed to be influenced by solvent pH or composition.

### DETAILED DESCRIPTION

### I. Abbreviations

Ca⁺²=calcium
CE=cholesterol esters
CH=cholesterol
Cl⁻=chloride
Co=cobalt
CISA=chloride ionic strength adjuster (for use with chloride ion selective electrode)
° C.=degrees centigrade
FA=fatty acids
Fe⁺²=iron
g=gram (1 g=1000 mg)
ICV=initial calibration verification sample
ISA=ionic strength adjuster (for use with calcium ion selective electrode)
ISE=ion selective electrode
L=liter (1 L=1000 mL)
M=molar
Mω==megohm
mg=milligram
mL=milliliter
mm=millimeter
mV=millivolt
μm=micrometer
N₂ gas=nitrogen gas

### NIST=National Institute of Standards and Technology

ppm=parts per million (1 ppm=1 mg/L)
SQ=squalene
SRM®=standard reference material
TG=triglycerides
W=tungsten
WC=tungsten carbide
WE=wax esters

### II. Explanation of Terms

“Amino acids” are acids that include an amino group, a carboxyl group, a hydrogen atom, and a distinctive R group bonded to a carbon atom. Some (but not all) amino acids are the structural units of proteins. The twenty amino acids encoded by the genetic code are called “standard amino acids.” These amino acids have the structure H₂N—CHR—COOH, where R is a side chain specific to the amino acids. Standard amino acids are alanine, arginine, aspargine, aspartic acid, cysteine, glutamic acid, glutamine, glycine, histidine, isoleucine, leucine, lysine, methionine, phenylalanine, proline, serine, theroinine, tryptophan, tyrosine, and valine. The amino acids citrulline and ornithine are non-standard amino acids that are not encoded by DNA and are not involved in protein synthesis, but are an intermediate in metabolic processes. In humans, the amino acids are present in the L-form, for example as L-Alanine (C₃H₇NO₂), L-(+)-Arginine (C₆H₁₄N₄O₂), L-(+)-Aspartic acid (C₄H₇NO₄), L-(+)-Citrulline (C₆H₁₃N₃O₃), L-(+)-Glutamic acid (C₅H₉NO₄), Glycine (C₂H₅NO₂), L-Histidine (C₆H₉N₃O₂), L-Isoleucine (C₆H₁₃NO₂), L-Leucine (C₆H₁₃NO₂), L-(+)-Lysine (for example in the form of L-Lysine-Monohydrochloride (C₆H₁₄N₂O₂.HCl)), L-(+)-Ornithine (for example as L-Ornithine Monohydrochloride (C₅H₁₂N₂O₂.HCl)), L-Phenylalanine (C₉H₁₁NO₂), L-Threonine (C₄H₉NO₃), L-(−)-Tryptophan (C₁₁H₁₂N₂O₂), L-Tyro sine (C₉H₁₁NO₃), and L-Valine (C₅H₁₁NO₂).

“Electrolytes” are ionized salts (ionic constituents) in body fluids that are generally capable of conducting an electrical current. The major electrolytic constituents in body fluids are made from sodium, potassium, magnesium, calcium, chloride, bicarbonate, and phosphate. Examples of electrolyte and ionic substances that may be dissolved in artificial human sweat include Sodium Sulfate (Na₂SO₄), Sodium Iodide (NaI), Sodium Fluoride (NaF), Sodium Bromide (NaBr), Cadmium Chloride Anhydrous (CdCl₂), Copper (II) Chloride Dihydrate (CuCl₂.2H₂O), Ammonium Hydroxide (NH₄OH), Sulfur (S), Iron Sulfate Heptahydrate (FeSO₄.7H₂O), Lead (Pb), Manganese (II), Chloride (MnCl₂), Nickel (Ni), Zinc (Zn), Sodium Bicarbonate (NaHCO₃), Potassium Chloride (KCl), Magnesium Chloride Hexahydrate (MgCl₂.6H₂O), Sodium Phosphate Anhydrous Monobasic (NaH₂PO₄), Calcium Chloride Dihydrate (CaCl₂.2H₂O), Phosphorous Pentachloride (PCl₅), and Sodium Chloride (NaCl).

A “nitrogenous substance” is a compound that contains a nitrogen, for example a nitrogen base. Examples of nitrogenous substances that may be found in human sweat include ammonium, uric acid (C₅H₄N₄O₃), urea (CH₄N₂O), creatinine (C₄H₇N₃O), and creatine (for example as creatine monohydrate (C₄H₉N₃O₂.H₂O)).

“Organic acid” is an acid made up of organic radicals, and which contains the ionizable COOH group. Examples of organic acids that may be found in human sweat include lactic acid (CH₃CH(OH)COONa), pyruvic acid (C₃H₄O₃), butyric acid (C₄H₈O₂), acetic acid (C₂H₄O₂), hexanoic acid (C₆H₁₂O₂), propionic acid (C₃H₆O₂), isobutyric acid (C₄H₈O₂), isovaleric acid (C₅H₁₀O₂), and D(+)-glucose (C₆H₁₂O₆).

“Vitamins” are essential low molecular weight organic compounds that are required in trace amounts for normal growth and metabolic processes; they often serve as components of coenzyme systems. Examples of vitamins that may be found in human sweat include thiamine (for example as thiamine hydrochloride (C₁₂H₁₇ClN₄OS.HCl)), riboflavin (C₁₇H₂₀N₄O₆), nicotinic acid (C₆H₅NO₂), pantothenic acid (for example as D-Pantothenic Acid (C₉H₁₇NO₅)), pyridoxine (for example as pyridoxine hydrochloride (C₈H₁₁NO₃.HCl)), folic acid (C₁₉H₁₉N₇O₆), ascorbic acid (for example as L-(+)-ascorbic acid (C₆H₈O₆) and/or dehydroascorbic acid), inositol (C₆H₁₂O₆), choline (for example as choline chloride (C₅H₁₄NOCl)), and aminobenzoic acid (for example as p-Aminobenzoic Acid (C₇H₇NO₂)).

The term “human sweat” is defined as non-exercise-induced eccrine (thermoregulatory) sweat secreted by healthy adult humans.

“Skin Surface Film Liquid” or “SSFL” encompasses historically used terms such as “artificial sweat,” “acidic artificial sweat,” “artificial perspiration,” “synthetic perspiration,” “synthetic sweat,” “sweat simulant,” and “simulated sweat.” The artificial SSFL disclosed herein contains both artificial sweat and artificial sebum components, which is contrary to the common understanding of prior SSFL compositions.

### III. Artificial SSFL Compositions

Human sweat is composed of highly variable amounts of primary electrolytes, ionic constituents, organic acids and carbohydrates, amino acids, nitrogenous substances, and vitamins and other miscellaneous constituents (Table 1). Sweat is 99.0 to 99.5% water and 0.5 to 1.0% solids (about half inorganic and half organic), with a specific gravity of 1.001 to 1.008. The composition of sweat can vary among people, and is also affected by age, diet, season, degree of acclimation, activity level, gender and race. The inventors therefore determined the range of components of sweat, and in disclosed embodiments provided a sweat formulation substantially having a mean concentration of most of the components of sweat for inclusion in a SSFL that would more closely mimic the formulation of the native liquid and its effect on items placed on the skin. This composition provides a superior dissolution medium for SSFL testing. The constituents of human sweat, and their median concentrations, are shown in Table 1.

TABLE 1
Median values of human sweat constituents

| Constituents | Concentration (M) |
|---|---|
| Primary electrolytes | |
| Sodium | 3.1 × 10⁻² |
| Chloride | 2.3 × 10⁻² |
| Calcium | 5.2 × 10⁻³ |
| Potassium | 6.1 × 10⁻³ |
| Magnesium | 8.2 × 10⁻⁵ |
| Phosphate | 3.1 × 10⁻⁴ |
| Bicarbonate | 3.0 × 10⁻³ |
| Ionic constituents | |
| Sulfate | 4.2 × 10⁻⁴ |
| Sulfur | 2.3 × 10⁻³ |
| Fluoride | 1.1 × 10⁻⁵ |
| Phosphorous | 1.3 × 10⁻⁵ |
| Bromine | 2.3 × 10⁻⁶ |
| Cadmium | 1.8 × 10⁻⁸ |
| Copper | 9.4 × 10⁻⁷ |
| Iodide | 7.1 × 10⁻⁸ |
| Iron | 9.8 × 10⁻⁶ |
| Lead | 1.2 × 10⁻⁷ |
| Manganese | 1.1 × 10⁻⁶ |
| Nickel | 4.2 × 10⁻⁷ |
| Zinc | 1.3 × 10⁻⁵ |
| Organic acids and carbohydrates | |
| Lactic acid | 1.4 × 10⁻² |
| Pyruvic acid | 1.8 × 10⁻⁴ |
| Butyric acid | 2.4 × 10⁻⁶ |
| Acetic acid | 1.3 × 10⁻⁴ |
| Hexanoic acid | 9.0 × 10⁻⁷ |
| Propionic acid | 3.5 × 10⁻⁶ |
| Isobutyric acid | 8.0 × 10⁻⁷ |
| Isovaleric acid | 1.1 × 10⁻⁶ |
| Glucose | 1.7 × 10⁻⁴ |
| Amino acids | |
| Alanine | 3.6 × 10⁻⁴ |
| Arginine | 7.8 × 10⁻⁴ |
| Aspartic acid | 3.4 × 10⁻⁴ |
| Citrulline | 4.0 × 10⁻⁴ |
| Glutamic acid | 3.7 × 10⁻⁴ |
| Glycine | 3.9 × 10⁻⁴ |
| Histidine | 5.2 × 10⁻⁴ |
| Isoleucine | 1.7 × 10⁻⁴ |
| Leucine | 2.1 × 10⁻⁴ |
| Lysine | 1.5 × 10⁻⁴ |
| Ornithine | 1.5 × 10⁻⁴ |
| Phenylalanine | 1.3 × 10⁻⁴ |
| Threonine | 4.5 × 10⁻⁴ |
| Tryptophan | 5.5 × 10⁻⁵ |
| Tyrosine | 1.7 × 10⁻⁴ |
| Valine | 2.5 × 10⁻⁴ |
| Nitrogenous Substances | |
| Ammonium | 5.2 × 10⁻³ |
| Uric acid | 5.9 × 10⁻⁵ |
| Urea | 1.0 × 10⁻² |
| Creatinine | 8.4 × 10⁻⁵ |
| Creatine | 1.5 × 10⁻⁵ |
| Vitamins | |
| Thiamine | 5.0 × 10⁻³ |
| Riboflavin | 2.0 × 10⁻² |
| Nicotinic Acid | 4.1 × 10⁻¹ |
| Pantothenic Acid | 1.3 × 10⁻¹ |
| Pyridoxine | 1.0 × 10⁻⁸ |
| Folic Acid | 1.6 × 10⁻⁸ |
| Ascorbic Acid | 1.0 × 10⁻⁵ |
| Dehydroascorbic Acid | 1.1 × 10⁻⁵ |
| Inositol | 1.6 × 10⁻⁶ |
| Choline | 2.6 × 10⁻⁵ |
| p-Aminobenzoic Acid | 7.1 × 10⁻⁸ |

The artificial SSFL compositions disclosed herein include both artificial sweat and artificial sebum. The artificial sweat formulations disclosed herein have one or more constituents (for example at least five or more constituents) from at least each of the categories of primary electrolytes, ionic constituents, organic acids and carbohydrates, and amino acids, for example at about the median concentrations shown in Table 1. In other embodiments, the artificial sweat formulation has all but five, three or one of the constituents in Table 1, and the constituents are present in about the median concentrations shown in that Table. In some embodiments the artificial sweat contains all of the listed constituents in substantially the median concentrations shown.

The artificial SSFL composition can, in some examples, further include a sebum formulation that includes squalene, wax esters, triglycerides, free fatty acids, cholesterol esters, free cholesterol and vitamin E in concentrations found in human sebum. For example, the sebum formulation may include 8-12% squalene, 23-27% wax esters, 30-35% triglycerides, 25-30% free fatty acids, 1-3% cholesterol esters, and 3-5% free cholesterol, and in more particular examples 10% squalene, 25% wax esters, 33% triglycerides, 28% free fatty acids, 2% cholesterol esters, and 4% free cholesterol. The wax esters may include palmityl palmitate and oleyl oleate; the triglycerides include tristearin and triolein; the free fatty acids include stearic, palmitic and oleic acid; the cholesterol esters include cholesteryl oleate; and the Vitamin E is (±)-α-tocopherol.

### IV. Methods of Making Artificial Sweat Compositions

Methods of making an artificial sweat composition are also disclosed, in which a volume of water of about 70-80% of a final desired liquid volume of the artificial sweat composition is provided. The primary electrolytes and ionic constituents are added to the water to form a primary solution, then the organic acids, amino acids, nitrogenous substances and vitamins are added to the primary solution.

In one disclosed embodiment, artificial sweat is prepared by combining the 60 constituents in the following five general classifications: primary electrolytes and other ionic constituents, organic acids and carbohydrates, amino acids, nitrogenous substances, and vitamins. For example, primary electrolytes (bicarbonate, sodium, calcium, chloride, manganese, phosphate, and potassium) and ionic constituents can be added first. Following addition of the electrolytes and ionic constituents, ionic concentrations can be measured, such as by using calcium and chloride ion selective electrodes. For example, the concentration of iron, calcium or chloride can be measured using semi-quantitative calorimetric test strips to determine if the measured values match that predicted from the weighed masses of the respective compounds. Organic acids (primarily in liquid form), followed by amino acids, nitrogenous substances, and vitamins can then be added to the solution. The artificial sweat composition can be maintained at a pH of 4.8-6.5, for example 5.0-5.5. In some examples, the sweat composition is stored at temperatures close to body temperature, for example 35-38° C., such as 36-37° C., to prevent or reduce precipitation and/or recrystallization of the constituents.

In a particular example, the electrolytes in the artificial sweat composition include sodium, chloride, calcium, potassium, magnesium, phosphate and bicarbonate; the ionic constituents include sulfate, sulfur, fluoride, phosphorous, bromine, cadmium, copper, iodide, iron, lead, manganese, nickel, zinc; the organic acids include lactic acid, pyruvic acid, butyric acid, acetic acid, hexanoic acid, propionic acid, isobutyric acid, and isovaleric acid; the carbohydrates include glucose; the amino acids include alanine, arginine, aspartic acid, citrulline, glutamic acid, glycine, histidine, isoleucine, leucine, lysine, ornithine, phenylalanine, threonine, tyrosine, and valine; the nitrogenous substances include ammonium, uric acid, creatinine and creatine; and the vitamins include thiamine, riboflavin, nicotinic acid, pantothenic acid, pyridoxine, folic acid, ascorbic acid, dehydroascorbic acid, inositol, choline and p-aminobenzoic acid, and the artificial sweat composition is maintained at a pH of 5.1-5.5.

In a more particular example, the electrolytes in the artificial sweat composition are present in a molar concentration of Na (3.0-3.5×10⁻²), Cl (2.0-2.5×10⁻²), Ca (5.0-5.5×10⁻³), K (5.8-6.2×10⁻³), Mg (1.0-1.5×10⁻⁴), PO₄ (2.8-3.4×10⁻⁴), and HCO₃ (2.7-3.3×10⁻³); the ionic constituents are present in a concentration of SO₄ (3.9-4.5×10⁻⁴), S (2.0-2.6×10⁻³), F (0.8-1.4×10⁻⁵), P (1.0-1.6×10⁻⁵), Br (2.0-2.6×10⁻⁶), Cd (1.5-2.1×10⁻⁸), Cu (9.1-9.7×10⁻⁷), I (6.8-7.4×10⁻⁸), Fe (9.5-10.1×10⁻⁶), Pb (0.9-1.5×10⁻⁷), Mn (0.8-1.4×10⁻⁶), Ni (3.9-4.5×10⁻⁷), and Zn (1.0-1.6×10⁻⁵); the organic acids and carbohydrates are present in concentrations of lactic acid (1.1-1.7×10⁻²), pyruvic acid (1.5-2.1×10⁻⁴), butyric acid (2.1-2.7×10⁻⁶), acetic acid (1.0-1.6×10⁻⁴), hexanoic acid (8.7-9.3×10⁻⁷), propionic acid (3.2-3.8×10⁻⁶), isobutyric acid (7.7-8.3×10⁻⁷), isovaleric acid (0.8-1.4×10⁻⁶), and glucose (1.4-2.0×10⁻⁴); the amino acids are present in concentrations of alanine (3.3-3.9×10⁻⁴), arginine (7.5-8.1×10⁻⁴), aspartic acid (3.1-3.7×10⁻⁴), citrulline (3.7-4.3×10⁻⁴), glutamic acid (3.4-4.0×10⁻⁴), glycine (3.6-4.2×10⁻⁴), histidine (4.9-5.5×10⁻⁴), isoleucine (1.4-2.0×10⁻⁴), leucine (1.8-2.4×10⁻⁴), lysine (1.2-1.8×10⁻⁴), ornithine (1.2-1.8×10⁻⁴), phenylalanine (1.0-1.6×10⁻⁴), threonine (4.2-4.8×10⁻⁴), tryptophan (5.2-5.8×10⁻⁵), tyrosine (1.4-2.0×10⁻⁴), and valine (2.2-2.8×10⁻⁴); the nitrogenous substances are present in a concentration of NH₃ (4.9-5.5×10⁻³), urea (0.7-1.3×10⁻²), uric acid (5.6-6.2×10⁻⁵), creatinine (8.1-8.7×10⁻⁵), and creatine (1.2-1.8×10⁻⁵); the vitamins are present in a concentration of thiamine (4.7-5.3×10⁻³), riboflavin (1.7-2.5×10⁻³), niacin (3.8-4.4×10⁻¹), pantothenic acid (1.0-1.6×10⁻¹), pyridoxine (0.7-1.3×10⁻⁸), folic acid (1.3-1.9×10⁻⁸), ascorbic acid (0.7-1.3×10⁻⁵) and/or its oxidation product dehydroascorbic acid (0.8-1.4×10⁻⁵), inositol (1.3-1.9×10⁻⁶), choline (2.3-2.9×10⁻⁵), and p-aminobenzoic acid (6.8-7.4×10⁻⁸).

In an even more particular example, artificial SSFL constituent concentrations match those of human sweat. The electrolytes are present in the artificial sweat composition in molar concentrations of Na (3.1×10⁻²), Cl (2.3×10⁻²), Ca (5.2×10⁻³), K (6.1×10⁻³), Mg (8.2×10⁻⁵), PO₄ (3.1×10⁻⁴), and HCO₃ (3×10⁻³); ionic constituents are present in a concentration of SO₄ (4.2×10⁻⁴), S (2.3×10⁻³), F (1.1×10⁻⁵), P (1.3×10⁻⁵), Br (2.3×10⁻⁶), Cd (1.8×10⁻⁸), Cu (9.4×10⁻⁷), I (7.1×10⁻⁸), Fe (9.8×10⁻⁶), Pb (1.2×10⁻⁷), Mn (1.1×10⁻⁶), Ni (4.2×10⁻⁷), and Zn (1.3×10⁻⁵); organic acids and carbohydrates are present in concentrations of lactic acid (1.4×10⁻²), pyruvic acid (1.8×10⁻⁴), butyric acid (2.4×10⁻⁶), acetic acid (1.3×10⁻⁴), hexanoic acid (9.0×10⁻⁷), propionic acid (3.5×10⁻⁶), isobutyric acid (8.0×10⁻⁷), isovaleric acid (1.1×10⁻⁶), and glucose (1.7×10⁻⁴); amino acids are present in concentrations of alanine (3.6×10⁻⁴), arginine (7.8×10⁻⁴), aspartic acid (3.4×10⁻⁴), citrulline (4.0×10⁻⁴), glutamic acid (3.7×10⁻⁴), glycine (3.9×10⁻⁴), histidine (5.2×10⁻⁴), isoleucine (1.7×10⁻⁴), leucine (2.1×10⁻⁴), lysine (1.5×10⁻⁴), ornithine (1.5×10⁻⁴), phenylalanine (1.3×10⁻⁴), threonine (4.5×10⁻⁴), tryptophan (5.5×10⁻⁵), tyrosine (1.7×10⁻⁴), and valine (2.5×10⁻⁴); the nitrogenous substances are present in concentrations of NH₃ (5.2×10⁻³), urea (1.0×10⁻²), uric acid (5.9×10⁻⁵), creatinine (8.4×10⁻⁵), and creatine (1.5×10⁻⁵); the vitamins are present in concentrations of thiamine (5.0×10⁻³), riboflavin (2.0×10⁻³), niacin (4.1×10⁻¹), pantothenic acid (1.3×10⁻¹), pyridoxine (1.0×10⁻⁸), folic acid (1.6×10⁻⁸), ascorbic acid (1.0×10⁻⁵) and/or its oxidation product dehydroascorbic acid (1.1×10⁻⁵), inositol (1.6×10⁻⁶), choline (2.6×10⁻⁵), and p-aminobenzoic acid (7.1×10⁻⁸); and the composition is at a pH of about 5.3.

### V. Methods of Making Artificial Sebum Formulations

Methods of making artificial sebum formulations are also disclosed herein. In some examples, sebum constituents are dissolved in a 2:1 co-solvent (chloroform:methanol). For example, sebum constituents including squalene, wax esters, triglycerides, free fatty acids, cholesterol esters, free cholesterol and vitamin E in concentrations as found in human sebum are dissolved in the 2:1 chloroform:methanol co-solvent solution.

In some examples, the sebum formulation include 8-12% squalene, 23-27% wax esters, 30-35% triglycerides, 25-30% free fatty acids, 1-3% cholesterol esters, and 3-5% free cholesterol, and in more particular examples 10% squalene, 25% wax esters, 33% triglycerides, 28% free fatty acids, 2% cholesterol esters, and 4% free cholesterol. The wax esters can also include palmityl palmitate and oleyl oleate; the triglycerides include tristearin and triolein; the free fatty acids include stearic, palmitic and oleic acid; the cholesterol esters include cholesteryl oleate; and the Vitamin E is (±)-α-tocopherol. In one example, the artificial sebum formulation is prepared to include at least 10% by mass of squalene; at least 20% wax esters; triglycerides; at least 25% free fatty acids; at least 1% cholesterol esters; at least 2% free cholesterol; and Vitamin E. In other embodiments, the sebum preparation includes about 10% squalene; 25% wax esters; 28% free fatty acids; 2% cholesterol esters; 4% free cholesterol; with the balance being triglycerides with a trace amount of Vitamin E.

In some examples, the prepared stock sebum solution can be stored at ambient atmosphere or under a nitrogen gas blanket and maintained at either room temperature (e.g., 23° C.) or at reduced temperature, such as less than 10° C. In a particular example, the stock sebum solution is maintained at 4° C. and under a nitrogen gas blanket. The artificial sweat and sebum formulations may be prepared and stored separately, and combined at the time of use, such as at the time of dissolution testing.

It is contemplated that additional methods known to one of skill in the art can be used to prepare the disclosed artificial sebum formulations, such as other solvent formulations (e.g., 3:1 chloroform:methanol) or microemulsion (e.g., Komesvarakul et al., J. Cosmet. Sci. 55: 309-325, 2006).

### VI. Methods of Combining Sweat and Sebum to Form Artificial SSFL

The exact mechanism by which sweat and sebum combine to form SSFL on human skin is not fully known, however several approaches can be used to formulate the disclosed artificial SSFL that contains both the artificial sweat and sebum. In some examples, artificial SSFL can be prepared by adding 500 mL of a disclosed artificial sweat composition at 35° C. to a molten mixture of a disclosed artificial sebum composition and stirring the two compositions, such as with a powerful whisk. In other examples, artificial SSFL can be prepared by emulsification.

Another approach to combining the artificial sweat and sebum is provided in the dissolution tests described in Example 9, in which a filter is coated with sebum and immersed in the artificial sweat to simulate SSFL on the surface of the skin. The sebum/sweat ratio can be varied to generate a sebum layer thickness ranging from approximately 0.05 to approximately 4 μm. In certain embodiments, the sebum is present on a mass basis in a ratio of at least or no more than 0/100, 1/99, 5/95, 10/90, 30/70, 50/50, 70/30, 90/10, 95/5, or 99/1. In one particular example, a mass basis of less than 1% sebum to 99% sweat can be used to generate a sebum layer thickness of approximately 2±1 μm

### VII. Methods of Performing Dissolution Tests with the Disclosed Compositions

Methods are disclosed for performing dissolution testing using the artificial sweat and sebum formulations either in combination (e.g., as artificial SSFL) or alone. Although dissolution testing in the combined sweat and sebum formulations provide particularly good results (as described below), testing can be performed separately with the sweat and sebum formulations to determine how these subcomponents of SSFL interact with particular test objects. Exemplary test methods include testing dissolution of an object (such as a metal, cloth or pharmaceutical object) in human sweat by exposing the object to the composition, including one of the disclosed sweat and/or sebum formulations, for a sufficient period of time for the composition to at least partially dissolve, then quantifying dissolution of the object in the composition.

A variety of in vitro test systems are available for assessing dissolution, including a static system and its variations (CEN, 1998; ISO, 2003), flow-through systems, and electrochemical systems. In some examples, a validated standard analytical method is employed with known reporting limits to minimize error associated with quantifying the mass of analyte that dissolves from a test article into the composition. For example, a limit of detection (LOD), defined as the mass of analyte that gives a mean signal three standard deviations above the mean blank signal, and/or a limit of quantification (LOQ), defined as the mass of analyte that gives a signal ten standard deviations above the mean blank signal, can be used. In certain examples, the composition including artificial SSFL or sweat/sebum alone is present at a volume so that the test article is completely immersed within the composition (such as at a ratio of 1 mL artificial SSFL per cm² of test article surface area). For test articles and materials with biologically relevant internal and external surface areas, a volume can be used that exposes either one or both the internal and external surfaces to the composition.

The duration of the in vitro dissolution studies can vary. In one example, an in vitro dissolution test is of sufficient length for the amount of material that dissolves to exceed the applicable analytical method reporting limit. For example, the duration can range from a few minutes to several days, such as from about one hour to about 120 days. In some examples, the duration of the dissolution is dependent upon whether the dissolution is mono-phasic (linear with time), biphasic (rapid initial phase followed by slower long-term phase), or multi-phasic (rapid initial phase, slower long-term phase, and a third very long-term phase). For example, time required for biphasic or multi-phase dissolution can be several weeks to months, depending on the characteristics of the test article.

The temperature of in vitro dissolution studies can also vary for the temperature of the composition can influence dissolution. For example, the temperature of the compositions can range from 10° C. to 100° C. In particular examples, the composition temperature is chosen to be within the range of the skin surface temperature of a human, such as from about 32.7 to 45.0° C. In an even more particular example, the composition is maintained at approximately 36.3° C. which is the median skin surface temperature calculated for resting adult humans.

In vitro dissolution tests can be performed with or without relative motion between the sample and one of the disclosed compositions. In dissolution systems with motion, the disclosed composition can be agitated to increase contact of the test article with the composition and to simulate real-world motion of the test article when in contact with human skin. In dissolution systems without motion, both the test article and composition remain static throughout the test. A variety of agitation techniques are available, including shaking using a mechanical shaker or wrist action shaker, stirring using a magnetic stirrer, ultrasonic agitation, and rotation.

The test liquids can contain varying ratios of sweat/sebum depending on study aims. On the skin, sebum spreads itself in a relatively thin, unevenly distributed sheet that ranges in thickness from less than 0.05 μm in sebum-poor areas to greater than 4 μm in sebum-rich areas such as the face. Sebum secretion rates vary among facial topographical regions, with climatic season, and with age. Thus, the ratio of sebum to sweat can be varied depending upon the desired thickness of sebum that is to be simulated.

In some embodiments, the disclosed artificial sebum is used alone without the artificial sweat, and in other embodiments the artificial sweat is used alone without the artificial sebum, or in the substantial absence of any other test compositions. For example, when modeling the release of a chemical (such as a dye) from materials in contact with the soles of the feet (rubber sandals, socks, etc.) where sebum glands are absent, a sweat-only formulation may be selected as more biologically relevant. In contrast, when modeling the dissolution of nanoparticles (such as zinc oxide used to coat fabrics for sun block applications) from cloth (such as a baseball cap or sun hat) in contact with the forehead, then a larger sebum component may be used to mimic the greater amounts of sebum present on the normal human forehead. For example, a large sebum component (on both a mass basis and thickness) can be achieved by placing the sebum composition directly onto the sample container rather than a filter.

The disclosed artificial sebum formulations can also be used alone to characterize the release and subsequent partitioning and absorption of chemicals from materials in contact with skin. For example, the influence of sebum on substance partitioning and absorption can be studied in various skin models using the disclosed artificial sebum. In one example, testing of partitioning in sebum alone can be evaluated by the protocol described by Valiveti et al. (Intern. J. Pharmaceutics 346:10-16, 2008) with our comprehensive sebum formulation. Exemplary skin models include pig skin, organotypic skin cultures (which do not contain sebaceous glands), neonatal rodent skin and shed snake skin.

### VIII. Benefits of a Comprehensive SSFL

The artificial SSFL disclosed herein has been found to have dissolution properties that are superior to currently widely used artificial sweat compositions. As illustrated in Example 10, cobalt powder dissolves to a much greater extent in the disclosed SSFL than it does in the artificial sweat composition used in the European CEN 1811 standard, which is the most widely used artificial sweat formulation today.

Prior formulations of artificial sweat often lacked many constituents that have now been determined to be present in human sweat. For example, among 45 formulations of artificial sweat in use between 1940 and 2005, seven lacked sodium, eight lacked chloride, 41 lacked calcium, 38 lacked potassium, 44 lacked magnesium, and all lacked bicarbonate. Of all ionic constituents in human sweat, only sulfate, fluoride, and phosphorous were included in several formulations of artificial sweat. All identified artificial formulations lacked sulfur, bromine, cadmium, copper, iodine, iron, lead, manganese, nickel, and zinc. A total of 16 amino acids have been found in human sweat; however, only four artificial formulations included more than one amino acid. No vitamins were included in any formulation of artificial sweat. Only five artificial SSFL formulations included a sebum component.

The disclosed artificial SSFL represents a comprehensive artificial sweat solution which includes 60 constituents identified in human sweat, as well as a representative sebum component. Of the 60 constituents, all but riboflavin were present in concentrations that corresponded to median values reported in human sweat. Despite containing 60 different constituents, including antioxidants such as ascorbic acid (vitamin C), this comprehensive artificial formulation (like all artificial formulations) lacks enzymatic free radical scavengers present in dynamic biological systems, e.g., Cu/Zn-super oxide dismutase and Mn-superoxide dismutase (Richelle et al., Br. J. Nutr. 96: 227-238, 2006).

The artificial sebum component used in the disclosed SSFL formulation includes nine constituents that fall into the six lipid categories identified in human sweat: squalene, wax esters, triglycerides, free fatty acids, cholesterol esters, and free cholesterol. The disclosed artificial sebum formulation differs from formulation previously disclosed, such as the formulation proposed by Musial and Kubis (Polim. Med. 34: 17-30, 2004) as follows: squalene content is lowered to better approximate the median value in human sebum (10.6%); lanolin wax esters are replaced with palmityl palmitate and oleyl oleate and the content raised to match the median value in human sebum (25%); animal-derived triglycerides are replaced with commercially available tristearin and triolein; free fatty acid content is raised to match the median value in human sebum (28.3%) and oleic acid is included as an unsaturated component; cholesterol esters as cholesteryl oleate are added at median value in human sebum (2%); and free cholesterol (4%) content is unchanged.

The disclosed artificial sebum formulation mimics the composition of human sebum by wt % of constituents. The presence of a representative sebum component coupled with a comprehensive sweat component that mimics its natural analogue provides a more robust SSFL than is currently available for investigating dissolution of chemicals in direct and prolonged contact with human skin. The disclosed artificial sebum formulations can also be used to characterize the release and subsequent partitioning and absorption of chemicals from materials in contact with skin. For example, the influence of sebum on substance partitioning and absorption can be studied in various skin models using the disclosed artificial sebum.

The subject matter of the present disclosure is further illustrated by the following non-limiting Examples.

### EXAMPLE 1

### Formulation of a Comprehensive Artificial Sweat

This example provides a method of preparing a comprehensive artificial sweat formulation.

All chemicals were ACS reagent grade, unless specified otherwise. Sodium sulfate, sodium iodide (certified grade), 1M ammonium hydroxide, sulfur (USP grade), lead reference solution (Certified grade), nickel reference solution (Certified grade), zinc reference solution (Certified grade), sodium bicarbonate, sodium phosphate anhydrous monobasic (USP grade), calcium chloride dihydrate, lactic acid, L-(+)-glutamic acid, 5N sodium hydroxide, oleic acid (NF grade), and methanol were from Fisher Scientific (Fair Lawn, N.J.). Sodium fluoride, sodium bromide, cadmium chloride anhydrous, copper(II) chloride dihydrate, iron sulfate heptahydrate, manganese(II) chloride, potassium chloride, phosphorous pentachloride, pyruvic acid, butyric acid, acetic acid, hexanoic acid, propionic acid, isobutyric acid, isovaleric acid, D(+)-glucose, DL-alanine, L-(+)-arginine, L-(+)-aspartic acid, L-(+)-citrulline, glycine, L-histidine, L-isoleucine, L-leucine, L-(+)-lysine monohydrochloride, L-(+)-ornithine monohydrochloride, L-phenylalanine, L-threonine, L-(−)-tryptophan, L-tyrosine, uric acid, urea, creatinine, creatine monohydrate, riboflavin, nicotinic acid, pyridoxine monochloride, L-(+)-ascorbic acid, inositol, choline chloride, squalene, stearic acid/palmitic acid, and chloroform were from Acros Organics (Geel, Belgium). Magnesium chloride hexahydrate, L-valine, thiamine monochloride, D-pantothenic acid, folic acid, ascorbate oxidase, p-aminobenzoic acid, tristearin (Technical grade), and Triolein were from MP Biomedicals, Inc. (Solon, Ohio). Sodium chloride, palmityl palmitate, oleyl oleate, cholesteryl oleate (Technical grade), cholesterol, and (±)-α-tocopherol (HPLC grade) were from Sigma-Aldrich (St. Louis, Mo.).

Calibrated chloride (Model 9617BN, Thermo Electron Corp., Beverly, Mass.) and calcium (Model 9320, Thermo Electron Corp.) ion selective electrodes were used to determine the stability and potential reactivity of representative constituents of the artificial sweat during preparation. After the addition of each class of chemicals, chloride and calcium concentrations were measured and compared to concentration values predicted from the masses of constituents dissolved during the preparation. A decrease in constituent concentration from mass-predicted values would indicate a reactive solution, whereas a reading corresponding to the predicted value would infer stability. These ions were chosen for their ability to be detected in a solution with high ionic strength, whereas other ionic-selective electrodes (e.g., fluorine, bromine, potassium) would have experienced interference from other ions present in solution. Iron strips (Aquacheck Total Iron Test Strips, Environmental Test Systems, Elkhart, Ind.) were used to semi-quantitatively monitor the stability and potential reactivity of iron in artificial sweat, especially in the presence of ascorbic acid (May et al., Biochem. Pharmacol. 57: 1275-1282, 1998).

To prepare one liter of the comprehensive artificial sweat, it was found to be helpful to start with a volume of water that was at least about 70-80% of the final desired volume of artificial sweat. This large initial volume was useful in promoting dissolution of the constituents of the artificial sweat. Hence to make a 1 L volume of artificial sweat, 800 mL of fully-aerated 18 megohm-cm distilled and deionized water was added to a 1000 mL Erlenmeyer flask, a magnetic stir bar was added to the flask, and the water was warmed to 36° C. using a stirrer/hotplate with a digital probe. Next, the desired masses of electrolytes and other ionic constituents (see Table 2) were added to the flask. Upon addition of the electrolytes and ionic constituents, the concentrations of chloride, calcium, and iron were measured to assess whether they matched predictions from weighing: 1.67×10⁻² M chloride, 6.93×10⁻³ M calcium, and 0.54 ppm iron. Next, organic acids and carbohydrates were added to the solution (see Table 2) and the stability of chloride, calcium, and iron concentrations verified. Amino acids were added (see Table 2) then calcium and iron concentrations verified; chloride ion concentration was measured to determine if it matched the predicted value (1.7×10⁻² M) from weighing electrolytes, ionic constituents, and hydrogen chloride salts of some amino acids. Nitrogenous substances were added (see Table 2) and chloride, calcium, and iron concentrations re-verified.

Vitamin constituents were added to the solution last (see Table 2). Dehydroascorbic acid was prepared by oxidizing an equal molar concentration of ascorbic acid (described in Example 2 below).

With the exception of riboflavin, nicotinic acid, and pantothenic acid, all vitamins dissolved rapidly in the solution. The concentration of riboflavin in human sweat ranges from 1.0×10⁻⁷ to 1.0 M, with a median 2.0×10⁻² M (Stefaniak and Harvey, Toxicol. In Vitro 20: 1265-1283, 2006). Due to the extremely low solubility of riboflavin (0.3 g/L water), only a maximum dissolved concentration of 2.0×10⁻³ M could be achieved, which is within the range reported for human sweat but a factor of 10 lower than the median value. This riboflavin concentration in artificial sweat was two orders of magnitude greater than that predicted by the solubility of riboflavin in water, an enhancement that may be due to solutropic interactions of dissolved nicotinic acid and urea (Coffman and Kildsig, J. Pharm. Sci. 85: 951-954. 1996) in the artificial sweat. It would be possible to achieve higher levels of dissolved riboflavin in artificial sweat using riboflavin salts such as riboflavin 5′-(dihydrogen phosphate), which has higher solubility (68 g/L water) at pH=5.6 than riboflavin. Riboflavin 5′-(dihydrogen phosphate) is endogenously present in humans as a cofactor in many enzymatic pathways. The excretion of riboflavin 5′-phosphate, however, occurs only after hydrolysis to free riboflavin (Jusko and Levy, J. Pharm. Sci. 56: 58-62, 1967) and is therefore not to be expected as a constituent in sweat, so it was not used. Upon addition of riboflavin and folic acid, the solution color changed from slightly yellow to a vivid yellow. The added quantities of pantothenic and nicotinic acids did not dissolve completely, which resulted in a cloudy solution.

After the addition of all vitamins, the concentrations of calcium and iron were re-verified; chloride ion concentration was measured to determine whether it matched the predicted value (2.2×10⁻² M) from weighing electrolytes, ionic constituents, and hydrogen chloride salts of some amino acids and vitamins. Finally, sodium and chloride levels were adjusted to match the median values in human sweat by addition of 5.8×10⁻² g sodium chloride per liter of solution and the concentrations of calcium and iron re-verified. Chloride concentration was measured to verify that it matched the final predicted value (2.3×10⁻² M) from weighing all constituent classes. The pH of the artificial sweat solution was raised from 3.8 (as prepared) to a final pH of 5.3 (as described in Example 3 below).

The final composition of the artificial sweat formulation is shown in Table 2.

TABLE 2
Comprehensive artificial sweat formulation

| Constituents | Mass (g/L) |
|---|---|
| Primary electrolytes and ionic constituents | |
| Sodium Sulfate (Na₂SO₄) | 5.83 × 10⁻² |
| Sodium Iodide (NaI) | 1.06 × 10⁻⁵ |
| Sodium Fluoride (NaF) | 4.62 × 10⁻⁴ |
| Sodium Bromide (NaBr) | 2.37 × 10⁻⁴ |
| Cadmium Chloride Anhydrous (CdCl₂) | 3.30 × 10⁻⁶ |
| Copper (II) Chloride Dihydrate (CuCl₂•2H₂O) | 1.60 × 10⁻⁴ |
| Ammonium Hydroxide (NH₄OH) | 1.82 × 10⁻¹ |
| Sulfur (S) | 7.37 × 10⁻² |
| Iron Sulfate Heptahydrate (FeSO₄•7H₂O) | 2.72 × 10⁻³ |
| Lead (Pb) - Reference Solution 1000 ppm | 2.49 × 10⁻⁵ |
| Manganese (II) Chloride (MnCl₂) | 1.38 × 10⁻⁴ |
| Nickel (Ni) - Reference Solution 1000 ppm | 2.46 × 10⁻⁵ |
| Zinc (Zn) - Reference Solution 1000 ppm | 8.50 × 10⁻⁴ |
| Sodium Bicarbonate (NaHCO₃) | 2.52 × 10⁻¹ |
| Potassium Chloride (KCl) | 4.55 × 10⁻¹ |
| Magnesium Chloride Hexahydrate (MgCl₂•6H₂O) | 1.67 × 10⁻² |
| Sodium Phosphate Anhydrous Monobasic (NaH₂PO₄) | 4.84 × 10⁻² |
| Calcium Chloride Dihydrate (CaCl₂•2H₂O) | 7.65 × 10⁻¹ |
| Phosphorous Pentachloride (PCl₅) | 2.71 × 10⁻³ |
| Sodium Chloride (NaCl)¹ | 5.84 × 10⁻² |
| Organic acids and carbohydrates | |
| Lactic acid (CH₃CH(OH)COONa) | 1.57 × 10⁰ |
| Pyruvic acid (C₃H₄O₃) | 1.59 × 10⁻² |
| Butyric acid (C₄H₈O₂) | 2.11 × 10⁻⁴ |
| Acetic acid (C₂H₄O₂) | 7.81 × 10⁻³ |
| Hexanoic acid (C₆H₁₂O₂) | 1.05 × 10⁻⁴ |
| Propionic acid (C₃H₆O₂) | 2.59 × 10⁻⁴ |
| Isobutyric acid (C₄H₈O₂) | 7.05 × 10⁻⁵ |
| Isovaleric acid (C₅H₁₀O₂) | 1.12 × 10⁻⁴ |
| D(+)-Glucose (C₆H₁₂O₆) | 3.06 × 10⁻² |
| Amino acids | |
| DL-Alanine (C₃H₇NO₂) | 5.11 × 10⁻² |
| L-(+)-Arginine (C₆H₁₄N₄O₂) | 1.36 × 10⁻¹ |
| L-(+)-Aspartic acid (C₄H₇NO₄) | 4.53 × 10⁻² |
| L-(+)-Citrulline (C₆H₁₃N₃O₃) | 7.01 × 10⁻² |
| L-(+)-Glutamic acid (C₅H₉NO₄) | 5.44 × 10⁻² |
| Glycine (C₂H₅NO₂) | 2.93 × 10⁻² |
| L-Histidine (C₆H₉N₃O₂) | 8.07 × 10⁻² |
| L-Isoleucine (C₆H₁₃NO₂) | 2.23 × 10⁻² |
| L-Leucine (C₆H₁₃NO₂) | 2.75 × 10⁻² |
| L-(+)-Lysine Monohydrochloride (C₆H₁₄N₂O₂•HCl) | 2.74 × 10⁻² |
| L-(+)-Ornithine Monohydrochloride (C₅H₁₂N₂O₂•HCl) | 2.53 × 10⁻² |
| L-Phenylalanine (C₉H₁₁NO₂) | 2.13 × 10⁻² |
| L-Threonine (C₄H₉NO₃) | 5.36 × 10⁻² |
| L-(−)-Tryptophan (C₁₁H₁₂N₂O₂) | 1.12 × 10⁻² |
| L-Tyrosine (C₉H₁₁NO₃) | 3.08 × 10⁻² |
| L-Valine (C₅H₁₁NO₂) | 2.93 × 10⁻² |
| Nitrogenous Substances | |
| Ammonium² | — |
| Uric acid (C₅H₄N₄O₃) | 9.92 × 10⁻³ |
| Urea (CH₄N₂O) | 6.01 × 10⁻¹ |
| Creatinine (C₄H₇N₃O) | 9.50 × 10⁻³ |
| Creatine Monohydrate (C₄H₉N₃O₂•H₂O) | 2.24 × 10⁻³ |
| Vitamins | |
| Thiamine Hydrochloride (C₁₂H₁₇ClN₄OS•HCl) | 1.69 × 10⁰ |
| Riboflavin (C₁₇H₂₀N₄O₆) | 7.53 × 10⁻¹ |
| Nicotinic Acid (C₆H₅NO₂) | 5.05 × 10¹ |
| D-Pantothenic Acid (C₉H₁₇NO₅) | 2.85 × 10¹ |
| Pyridoxine Hydrochloride (C₈H₁₁NO₃•HCl) | 2.06 × 10⁻⁶ |
| Folic Acid (C₁₉H₁₉N₇O₆) | 7.06 × 10⁻⁶ |
| L-(+)-Ascorbic Acid (C₆H₈O₆) | 1.76 × 10⁻³ |
| Dehydroascorbic Acid | 1.91 × 10⁻³ |
| Inositol (C₆H₁₂O₆) | 2.88 × 10⁻³ |
| Choline Chloride (C₅H₁₄NOCl) | 3.63 × 10⁻³ |
| p-Aminobenzoic Acid (C₇H₇NO₂) | 9.73 × 10⁻⁶ |

¹Added last to adjust sodium and chloride content of artificial sweat formulation to match median values in human sweat
²Added as ammonium hydroxide with primary electrolytes and ionic constituents

### EXAMPLE 2

### Preparation of Dehydroascorbic Acid from Ascorbic Acid

This example provides a method of preparing dehydroascorbic acid from asorbic acid.

To prepare dehydroascorbic acid calibration standards, six 100 mL solutions of ascorbic acid were prepared at concentrations that ranged from 1×10⁻²M through 1×10⁻⁷ M, using decade dilutions. The ascorbic acid solutions were completely oxidized to dehydroascorbic acid by adding 200 μL of the enzyme ascorbate oxidase to each solution, then incubating at 37° C. for 30 minutes. Using a spectrophotometer (Spectronic 20 Genesys, Spectronic Instruments, Inc., Rochester, N.Y.), a calibration curve was prepared by measuring the absorbance of the dehydroascorbic acid standard solutions at 346 nm.

To prepare one liter of artificial sweat that contained dehydroascorbic acid at the median concentration in human sweat, 1.7×10⁻² g ascorbic acid was dissolved in 100 mL of 18 megohm-cm distilled and deionized water. Next, 200 μL of ascorbate oxidase was added to the solution and it was incubated for 30 minutes at 37° C., followed by verification of the concentration of dehydroascorbic acid using the calibrated spectrophotometer. Note that after verifying the concentration of dehydroascorbic acid, the solution can be incubated at 37° C. for three days to render the ascorbate oxidase inactive, thus eliminating the possibility of oxidizing ascorbic acid present in sweat.

### EXAMPLE 3

### Artificial Sweat Solution pH

This example provides a method for increasing the pH of an artificial sweat solution.

The pH of the artificial sweat solution was raised from 3.8 (as prepared) to a final pH of 5.3 by adding 5 M sodium hydroxide dropwise while monitoring using a calibrated pH electrode (InLab 413, Mettler-Toledo, Schwerzenbach, Switzerland) connected to a multi-meter (Seven Multi, Mettler-Toledo). Use of 5 M sodium hydroxide caused any undissolved vitamins to go completely into solution. The final volume of the solution was then brought up to 1.0 L using 18 megohm-cm distilled and deionized water and the final pH verified. To minimize opportunity for microbial growth, the final sweat solution was filtered through a 0.2 μm pore size filter (Cat. No. SCGVU11RE, Millipore Corp., Billerica, Mass.). Additional studies demonstrated the sweat formulation could be adjusted to a pH up to 6.5 while maintaining the integrity of the solution. Additionally, the batch volume has been successfully scaled up from 1.0 L to 10.0 L.

### EXAMPLE 4

### Formulation of an Artificial Sebum

This example provides a method of preparing an artificial sebum formulation.

Artificial sebum was prepared by dissolving nine different lipid constituents (see Table 3) in a sterilized 1000 mL Erlenmeyer flask that contained 500 mL of a 2:1 co-solvent mixture of chloroform and methanol while stirring continuously using a magnetic stir bar and stir plate.

TABLE 3
Artificial sebum formulation

| Constituent | Saturation | Chemical | Mass (g) |
|---|---|---|---|
| Squalene | | Squalene | 0.5151 |
| Wax esters | Saturated | Palmityl Palmitate | 0.9718 |
| | Unsaturated | Oleyl Oleate | 0.2430 |
| Triglycerides | Saturated | Tristearin | 1.0690 |
| | Unsaturated | Triolein | 0.5345 |
| Free Fatty Acids | Saturated | Stearic/Palmitic Acids | 0.6876 |
| | Unsaturated | Oleic Acid | 0.6876 |
| Cholesterol Esters | | Cholesteryl Oleate | 0.0972 |
| Free Cholesterol | | Cholesterol | 0.1944 |
| Vitamin E | | (±)-α-Tocopherol | 0.0050 |

### EXAMPLE 5

### Characterization of the Stability and Shelf-Life of Artificial Sweat and Artificial Sebum Formulations

This example demonstrates that the optimal storage conditions for the disclosed artificial sweat and sebum formulations as well as the shelf-life of the disclosed artificial SSFL.

Dissolution of fine materials (e.g., particles) is often evaluated by isolating the test material between two filters in a dissolution chamber, then immersing the chamber in a model liquid. Thus, to characterize the stability of artificial sweat and artificial sebum in combination, the sebum component was deposited onto mixed cellulose ester filters by immersing each filter into the sebum solution, then allowing the chloroform:methanol co-solvent to evaporate off at ambient temperature and atmosphere. Alternatively, for large test articles, the artificial sebum could be deposited directly onto the walls of the container used to house the solvent and test article. It was assumed that artificial sebum was evenly deposited on the top and bottom surfaces of each filter. Modeling filters as a geometric cylinder, the thickness of artificial sebum was estimated to be 1.0 μm, which is consistent with the thickness of human sebum on skin. Thickness calculations were verified by visual inspection of uncoated and sebum-coated filters using a scanning electron microscope (SEM). In the SEM, the artificial sebum appeared as a hazy coating of approximately 1 μm at the edge of the filter. Each sebum-coated filter was immersed in 80-mL artificial sweat contained in a polypropylene plastic cup with screw cap lid, then pH, chloride, calcium, and iron values were monitored twice daily for 14 days as previously described.

The stability of the novel artificial SSFL sweat and sebum components was tested in combination and individually. To characterize the stability of sweat and sebum in combination, 47-mm mixed cellulose ester filters were coated with a 1 μm thick layer of artificial sebum. On human skin, sebum forms a 0.5 to >4.0 μm thick layer. Each sebum-coated filter was immersed in 80 mL of artificial sweat maintained at 36.3° C., the median temperature reported for human skin. The pH, [Cl⁻], [Ca⁺²], and [Fe] of the artificial sweat, monitored twice daily for two weeks were: 5.3±0.1, 3.0±0.1×10⁻² M, 7.4±0.1×10⁻³ M, and 0.5 ppm, respectively at 36.3° C.

The stock artificial sweat and sebum formulations were also characterized individually under different storage conditions to estimate shelf life. The pH, chloride, calcium, and iron values of the stock artificial sweat were monitored at room temperature (23° C.) daily for 14 days. The [Cl⁻], [Ca⁺²], and [Fe] levels of the artificial sweat remained equivalent to median values in human sweat over a two week characterization period (data not shown) when stored at room temperature (23° C.). During this same period, the artificial sweat had a mean pH of 5.3±0.1 at 23° C. The pH of human sweat ranges from 2.1 to 8.2, with a median value of 5.3, but varies due to endogenous (e.g., age, ethnicity, sebum content, sweat content) and exogenous (e.g., use of cosmetics, soaps, occlusive dressings) factors. The formulation of comprehensive sweat described herein has a pH that mimics the median value of human sweat; however, this formulation may not be suitable for analyzing the behavior of materials in artificial SSFL under more acidic conditions. If the pH of this artificial sweat formulation was below 4.8, precipitation of salts occurred rapidly.

Monitoring of the pH of the stock artificial sweat solution was stopped on day 14; however, the solution was visibly unchanged until day 18 when microbial growth appeared on the surface of the liquid. Stability of the artificial sweat at ambient temperature may be enhanced by filtering the liquid to remove bacteria and ensuring the sterility of all glassware used to transfer and store the sweat. Filtering the artificial sweat with a 0.2 micron pore filter is an example of a filtration method that substantially removes the bacteria. Refrigeration of the artificial sweat is not recommended; when stored at 4° C., precipitation and/or recrystallization of constituents occurred. Hence in certain disclosed methods, the sweat composition is stored while being maintained at temperatures close to body temperature, for example 35-38° C., such as 36-37° C.

Initially, stability of the stock artificial sebum was evaluated by visually monitoring solution color and clarity over a two week period. Two storage conditions for the sebum solution were used: ambient atmosphere and temperature versus displacement of air in the flask headspace with nitrogen gas and refrigeration at 4° C. When stored at room temperature and atmosphere, the sebum solution oxidized in less than 24 hours as indicated by a color change from slightly white to yellow, presumably due to the oxidation of triolein. To inhibit oxidation of the prepared stock sebum solution, the flask was evacuated and the solution blanketed with nitrogen, then stored at 4° C. When stored under these latter conditions, the stock sebum appeared to be stable for over two weeks. Subsequent quantitative studies were performed using thin layer chromatography and confirmed that the sebum constituents maintained dry or in the chloroform:methanol solution at 4° C. were stable through 28 days in the presence and absence of vitamin E. In the presence of vitamin E, the artificial sebum formulation could be used for 28 days at 23° C., a temperature similar to human skin (Example 10).

These studies demonstrate that the disclosed artificial SSFL formulation was a chemically-stable artificial SSFL with a well-buffered pH that can be used in studies of the potential release and percutaneous absorption of chemicals that dissolve from articles in contact with skin. These studies also demonstrate that optimal storage conditions for the disclosed artificial sebum formulation include storage under a nitrogen blanket at a reduced temperature (e.g., 4° C.).

### EXAMPLE 6

### Preparation of Artificial Sweat

This example provides a detailed protocol for preparing artificial sweat.

The artificial sweat formulation was prepared by providing 80% of the desired final volume of 18MΩ distilled and deionized water to a large carboy (4.8 L water for 6 L batch or 7.2 L water for 9 L batches). A stir bar and temperature probe were placed in the container then stirring begun at 450 rpm on a magnetic stir plate while warming to 36.3° C. (Model 11-800-495SHP, Fisher Scientific, Dubuque, Iowa). The inlet side of an in-line HEPA filter (HEPA-CAP 36, Whatman Inc., Florham Park, N.J.) was connected to an air supply and an aeration stone was connected to the outlet side of the filter. An aeration stone was placed in the water of the carboy and air bubbled into the liquid for 1 hour to saturate it with air.

A weighing dish was placed on a calibrated microbalance (Model XS205, Mettler-Toledo, Greifensee, Switzerland). Each constituent (beginning with sodium sulfate) was weighed out or pipeted in the appropriate mass or volume of each constituent, and the weighed mass of the constituent recorded. The weighed mass was then transferred to the appropriate carboy then the weighing dish rinsed with water to ensure complete transfer into beaker. Each of the constituents was transferred in this manner, in the order in listed in Table 4 below.

The mass or volume of some chemical constituents in the table were too small to be weighed with a desired accuracy on the balance or dispensed by the pipets, so the protocol was modified by preparation of concentrated solutions that were pipetted into a beaker:

a. Sodium Iodide: Dissolve 0.010 g NaI in 0.050 L water
b. Sodium Fluoride: Dissolve 0.020 g NaF in 0.050 L water
c. Sodium Bromide: Dissolve 0.010 g NaBr in 0.050 L water
d. Cadmium Chloride Anhydrous: Dissolve 0.010 g CdCl₂ in 0.050 L water
e. Copper Chloride Dihydrate: Dissolve 0.010 g CuCl₂.2H₂O in 0.050 L water
f. Lead Reference Solution: Pipet 1.0 mL lead reference solution in 0.050 L water
g. Manganese Chloride: Dissolve 0.010 g MnCl₂ in 0.050 L water
h. Nickel Reference Solution: Pipet 1.0 mL nickel reference solution in 0.050 L water
i. Hexanoic Acid: Pipet 1.0 mL hexanoic acid in 0.050 L water
j. Isobutyric Acid: Pipet 1.0 mL isobutyric acid in 0.050 L water
k. Isovaleric Acid: Pipet 1.0 mL isovaleric acid in 0.050 L water
l. Creatinine: Dissolve 1.000 g creatinine in 0.250 L water
m. Creatine monohydrate: Dissolve 1.000 g creatinine monohydrate in 0.250 L water
n. Pyridoxine Hydrochloride: Dissolve 0.010 g pyridoxine hydrochloride in 0.050 L water
o. Folic Acid: Dissolve 0.010 g folic acid in 0.050 L water
p. Ascorbic Acid: Dissolve 0.050 g ascorbic acid in 0.050 L water
q. Inositol: Dissolve 0.010 g inositol in 0.050 L water
r. Choline Chloride: Dissolve 1.000 g choline chloride in 0.050 L water
s. p-Aminobenzoic Acid: Dissolve 0.010 g p-aminobenzoic acid in 0.050 L water

The only modification of this procedure was for addition of the vitamins, in which the protocol was modified by placing 50 mL of 5N sodium hydroxide in a 100 mL beaker and stirring it at 450 rpm, then adding pantothenic acid to the beaker until the solution was milky in appearance (˜10 mg). The remaining pantothenic acid powder was added in small increments to the 2 L beaker containing the sweat solution and went into solution. Then the riboflavin was added to the 2 L beaker, whereupon the solution turned orange-yellow. Next, the sodium hydroxide/pantothenic acid solution was added to the 2 L beaker in aliquots of ˜10 mL. To rinse the 100 mL beaker, the remaining vitamins (in liquid form) were added to the beaker along with 50 mL dehydroascorbic acid. The solution was transferred to the 2 L beaker with the sweat then rinsed a second time with 50 mL DHA. Finally, nicotinic acid was added incrementally (˜10 mg at a time) and solution began to clear as constituent was added. The stirrer was turned off, then pH was raised to 5.3 by adding sodium hydroxide 1 mL at a time with stirring after each addition of base. After addition of the vitamins, the warm solution was stirred overnight.

TABLE 4
Preparation of artificial sweat (pH 5.3)

| Sweat Constituent | Mass (g/L) | Volume (μL/L) |
|---|---|---|
| Primary electrolytes and ionic constituents | | |
| Sodium Sulfate (Na₂SO₄) | 0.05826 | — |
| Sodium Iodide (NaI) | — | 54.6 |
| Sodium Fluoride (NaF) | — | 2310.9 |
| Sodium Bromide (NaBr) | — | 1185.6 |
| Cadmium Chloride Anhydrous (CdCl₂) | — | 16.4 |
| Copper (II) Chloride Dihydrate (CuCl₂•2H₂O) | — | 803.4 |
| 1M Ammonium Hydroxide (NH₄OH) | — | 186.0 |
| Sulfur (S) | 0.07374 | — |
| Iron Sulfate Heptahydrate (FeSO₄•7H₂O) | 0.00272 | — |
| Lead (Pb) - Reference Solution 1000 ppm | — | 1243.0 |
| Manganese (II) Chloride (MnCl₂) | — | 691.4 |
| Nickel (Ni) - Reference Solution 1000 ppm | — | 1232.0 |
| Zinc (Zn) - Reference Solution 1000 ppm | — | 0.77 |
| Sodium Bicarbonate (NaHCO₃) | 0.25203 | — |
| Potassium Chloride (KCl) | 0.45469 | — |
| Magnesium Chloride Hexahydrate (MgCl₂•6H₂O) | 0.01667 | — |
| Sodium Phosphate Anhydrous Monobasic (NaH₂PO₄) | 0.04836 | — |
| Calcium Chloride Dihydrate (CaCl₂•2H₂O) | 0.76450 | — |
| Phosphorous Pentachloride (PCl₅) | 0.00271 | — |
| Sodium Chloride | 0.05844 | — |
| Organic acids and carbohydrates | | |
| Sodium Lactate (CH₃CH(OH)COONa) | — | 2011.0 |
| Pyruvic acid (C₃H₄O₃) | — | 12.7 |
| Butyric acid (C₄H₈O₂) | — | 0.22 |
| Acetic acid (C₂H₄O₂) | — | 7.43 |
| Hexanoic acid (C₆H₁₂O₂) | — | 5.70 |
| Propionic acid (C₃H₆O₂) | — | 0.26 |
| Isobutyric acid (C₄H₈O₂) | — | 3.70 |
| Isovaleric acid (C₅H₁₀O₂) | — | 6.00 |
| D(+)-Glucose (C₆H₁₂O₆) | 0.03063 | — |
| Amino acids | | |
| DL-Alanine (C₃H₇NO₂) | 0.05114 | — |
| L-(+)-Arginine (C₆H₁₄N₄O₂) | 0.13588 | — |
| L-(+)-Aspartic acid (C₄H₇NO₄) | 0.04525 | — |
| L-(+)-Citrulline (C₆H₁₃N₃O₃) | 0.07008 | — |
| L-(+)-Glutamic acid (C₅H₉NO₄) | 0.05444 | — |
| Glycine (C₂H₅NO₂) | 0.02927 | — |
| L-Histidine (C₆H₉N₃O₂) | 0.08068 | — |
| L-Isoleucine (C₆H₁₃NO₂) | 0.02230 | — |
| L-Leucine (C₆H₁₃NO₂) | 0.02755 | — |
| L-(+)-Lysine Monohydrochloride (C₆H₁₄N₂O₂•HCl) | 0.02740 | — |
| L-(+)-Ornithine Hydrochloride (C₅H₁₂N₂O₂•HCl) | 0.02529 | — |
| L-Phenylalanine (C₉H₁₁NO₂) | 0.02148 | — |
| L-Threonine (C₄H₉NO₃) | 0.05360 | — |
| L-(−)-Tryptophan (C₁₁H₁₂N₂O₂) | 0.01123 | — |
| L-Tyrosine (C₉H₁₁NO₃) | 0.03080 | — |
| L-Valine (C₅H₁₁NO₂) | 0.02928 | — |
| Nitrogenous Substances | | |
| Ammonium¹ | — | |
| Uric acid (C₅H₄N₄O₃) | 0.00992 | — |
| Urea (CH₄N₂O) | 0.60060 | — |
| Creatinine (C₄H₇N₃O) | — | 1357.0 |
| Creatine Monohydrate (C₄H₉N₃O₂•H₂O) | — | 447.8 |
| Vitamins | | |
| Thiamine Hydrochloride (C₁₂H₁₇ClN₄OS•HCl) | 1.68630 | — |
| Riboflavin (C₁₇H₂₀N₄O₆) | 0.7527 | — |
| Nicotinic Acid (C₆H₅NO₂) | 50.47510 | — |
| D-Pantothenic Acid (C₉H₁₇NO₅) | 28.50120 | — |
| Pyridoxine Hydrochloride (C₈H₁₁NO₃•HCl) | — | 10.3 |
| Folic Acid (C₁₉H₁₉N₇O₆) | — | 35.3 |
| L-(+)-Ascorbic Acid (C₆H₈O₆) | — | 877.2 |
| Dehydroascorbic Acid² | — | 100.0 mL/L |
| Inositol (C₆H₁₂O₆) | — | 1441.4 |
| Choline Chloride (C₅H₁₄NOCl) | — | 726.3 |
| p-Aminobenzoic Acid (C₇H₇NO₂) | — | 47.3 |

¹Added as ammonium hydroxide with primary electrolytes and ionic constituents
²Use stock solution stored in 4° C. refrigerator

To verify the final composition of the artificial sweat, the expected chloride (Cl⁻) concentration was calculated in each of the three artificial sweat solutions using a chloride ion selective electrode (Cl⁻ ISE) (Model 9617BN, Thermo Electron Corp) connection to mV meter (Model AR15, Fisher Scientific). The expected calcium concentration was also calculated and measured using a calcium ion selection electrode (Ca⁺² ISE) (Model 9320, Thermo Electron Corp) connection to mV meter (Model AB15, Fisher Scientific). The iron concentration was calculated and then measured by adding one foil packet of iron reducing powder pillows (AquaChek® total iron test strips, Environmental Test Systems, Elkhart, Ind.) to each vial with artificial sweat solution. The vial was capped and shaken for 5 seconds or until powder dissolves. A test strip was dipped in the solution and compared to a color chart on the test strip bottle.

### EXAMPLE 7

### Preparation of Sebum Coated Filters for Dissolution Study

This example provides a protocol for preparing sebum coated filters for use in dissolution studies.

The sebum solution in an organic solvent (lipids dissolved in 2:1 chloroform methanol) was removed from a 4° C. refrigerator and placed on a hotplate/stirrer. Valves were opened on the container to purge nitrogen gas in the flask, and the solution was gently warmed until the solution turned clear and all solids dissolved. Whatman 541 ashless filter circles were weighed and placed on a screen of a uniquely identified dissolution chamber. After the sebum had cleared, 50 mL of solution were poured into a clean wide-mouth beaker, and each Whatman 541 ashless filter circle was dipped in the sebum with tweezers then placed on top of a screen in a dissolution chamber. Solvent was allowed to evaporate for 30 minutes to yield sebum on filter; each sebum-coated filter was weighed to determine the weight increase.

When finished with the sebum solution, its flask headspace was purged and replaced with N₂ gas to inhibit oxidation of lipids. Purging was performed by stoppering the flask and closing all valves. One end of the tubing was connected to a vacuum for a few seconds and the valve then closed. The tubing was then connected at one end to vacuum and at the other end to an N₂ gas canister. The flask of sebum solution was then refrigerated at 4° C.

### EXAMPLE 8

### Preparation of Filter Sandwiches for Dissolution Study of Hard Metals

This example provides a protocol for preparing filter sandwiches for use in dissolution studies.

A sebum-coated 47 mm Whatman 541 ashless filter circle was placed on top of a 47 mm nitrocellulose filter having a 0.025-μm pore size. The two filters (with the sebum coated filter on top) were placed on the screen resting in the lower ring of the dissolution chamber (Cat. No. 06-401, In-Tox Products, LLC, Moriarity, N. Mex.).

The filters and chamber half on the microbalance were tared. Using a clean spatula, the required minimum mass of bulk or size-separated powder to be dissolution tested was added to the sebum-coated filter.

As illustrated in Table 5, powder masses were adjusted to yield equivalent masses of cobalt in all samples; the mass of tungsten will be equivalent in the W (tungsten), WC (tungsten carbide), and add-mix powders, but not the spray dryer or chamfer material.

TABLE 5
Powder masses of hard metal powder samples.

| | Powder Mass (g) | | | | | |
|---|---|---|---|---|---|---|
| Size | W | WC | Co | Admix | Spray dry | Chamfer |
| Bulk | 0.0460 | 0.0489 | 0.0032 | NA | NA | NA |
| Stage 1 | 0.0460 | 0.0489 | 0.0032 | 0.0489 | 0.0252 | 0.0278 |
| Stage 2 | 0.0460 | 0.0489 | 0.0032 | 0.0489 | 0.0252 | —^{a} |
| Stage 3 | 0.0460 | 0.0489 | 0.0032 | 0.0489 | — | — |

^{a}Insufficient mass of material for planned dissolution study

The actual mass of powder for each sample was recorded, and a second 0.025-μm pore nitrocellulose filter was placed on top of the powder and sebum-coated Whatman 541 to encase the material. An O-ring was placed on top of the filter assembly, the top half of the chamber was seated on the assembly, and the assembly was screwed together using 4 nylon screws to form a particle-tight seal around the perimeter of the filter sandwich. Each static dissolution chamber was stored in a clean, re-sealable plastic bag that was marked with the contents and sample number.

Additional filter sandwiches were made without powder at a ratio of 1 in 10 for use as blanks. One undosed sandwich was placed into a static dissolution chamber then stored in a clean, re-sealable plastic bag. Bulk tungsten, tungsten carbide, or cobalt powder was used to spike filter sandwiches containing known amount of particles (using the same masses as experimental samples) at a ratio of 1 in 10 per material type.

### EXAMPLE 9

### Dissolution Testing in Artificial SSFL

This example demonstrates dissolution testing in artificial SSFL.

A 0.22 μm pore filter was used to filter 1 L of artificial sweat solution using a sterile 1 L filter unit and vacuum. With a sterile pipet, 80 mL of artificial sweat was added to each appropriately labeled autoclaved polypropylene jar with a screw-top lid. The lid was screwed on each jar and all jars were placed in the incubator at 36.3° C. for 3 hours to equilibrate solvent temperature. After 3 hours, the jars were removed one at a time from the incubator, the lid unscrewed, a static dissolution chamber placed in the artificial sweat. For each solvent formulation, triplicate samples of cobalt powder were studied. Using a sterile pipet, 80 mL of artificial sweat was added to the empty autoclaved polypropylene jars and placed in the incubator to equilibrate solvent prior to the next scheduled change out. The container was covered and returned to the incubator.

At 10 pre-determined time points (1, 4, 8, 12, 16, 20, 24, 32, 40, and 48 hours) samples of solvent from each container were collected for analysis of dissolved cobalt and tungsten concentration. The purpose of measuring masses of dissolved cobalt and tungsten at multiple time points was to investigate the dissolution kinetics of these powders. At each time point, a sample jar and a jar containing equilibrated solvent were removed one at a time from the incubator and the lids unscrewed. Using plastic forceps, the dissolution chamber was removed from the solvent and shaken to remove excess liquid. The powder-exposed liquid was transferred into a clean numbered borosilicate glass sample jar and a record made of the sample contents, time, and any other pertinent data. Each borosilicate glass jar was maintained at a temperature of ˜36° C. The dissolution chamber was returned to its static dissolution chamber in its appropriate container, immersed in fresh temperature-equilibrated solvent, covered with the lid, and returned to the incubator. These steps were repeated until the solvent had been changed in all samples.

The pH of solvent in each borosilicate glass sample jar was measured at 36.3° C. After determining the pH of all samples, the jars were placed in a −80° C. freezer for storage until analysis by spectroscopy.

The quality control samples were treated in a similar manner using the filter sandwiches containing sebum-coated filters at a ratio of 1 in 10 prepared dissolution chambers. The artificial sweat was added at the same pre-determined time points as the experimental samples. Samples were spiked with tungsten and cobalt powder at a ratio of 1 in 10 prepared dissolution chambers.

All experimental and quality control samples were submitted blind (without identification of the sample type or study design) to a commercial laboratory for quantification of cobalt content. Blinded quality control samples included field blank samples, reagent blank samples (artificial SSFL, artificial CEN sweat, or filters). All samples were analyzed by US Occupational Safety and Health Administration (US OSHA) Method ID-213: Tungsten and cobalt in workplace atmospheres (ICP analysis), a fully-validated standard method for quantification of soluble and insoluble cobalt. Artificial SSFL and CEN sweat samples, which contained dissolved cobalt and/or tungsten, were analyzed without digestion to quantify the mass of dissolved metals (MD). The matrix-specific method limits of detection (LOD) and limits of quantification (LOQ) for liquid samples are provided in Table 6.

TABLE 6
Matrix-specific method LOD and LOQ for liquid samples

Co (mg/L)

| Formulation | LOD | LOQ |
|---|---|---|
| NIOSH | 0.6 | 2 |
| CEN | 0.3 | 0.83 |

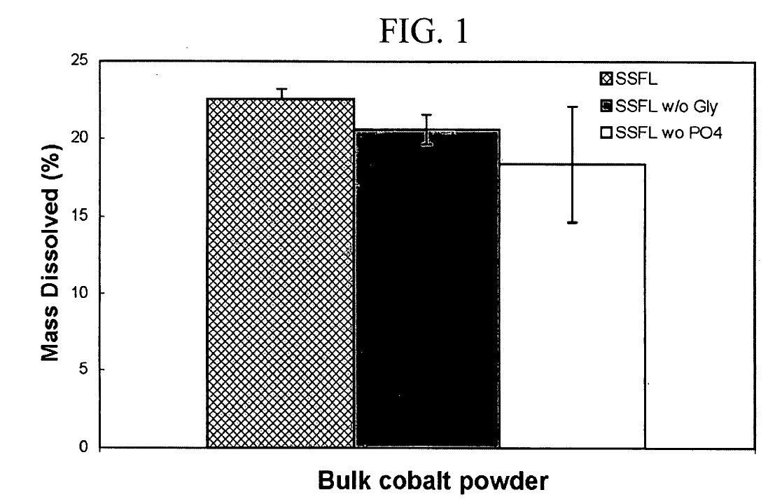

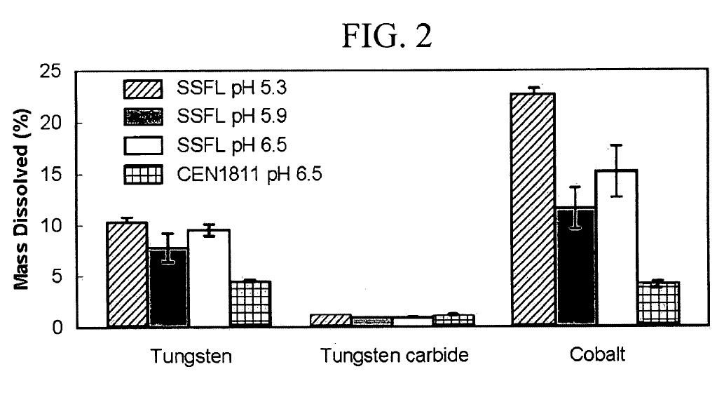

FIG. 1 summarizes the total masses of cobalt dissolved during the 48 hour dissolution study using three variations of artificial SSFL (full formulation, without glycine, and without phosphate). One-way analysis of variance analysis revealed that dissolution rates were similar among these three variations of artificial SSFL (p=0.16). FIG. 2 summarizes the total masses of tungsten dissolved from tungsten metal powder and tungsten carbide powder and the total mass of cobalt dissolved from cobalt metal powder in three variations of the full formulation of artificial SSFL (pH 5.3, 5.9, and 6.5) and the European Committee for Standardization (CEN) artificial sweat formulation described in EN 1811, which is a simple solution of 0.5% NaCl, 0.1% urea and 0.1% lactic acid, with a pH adjusted to 6.5 using NH₄OH.

On a mass basis, the percentage of tungsten powder dissolved in SSFL at all pH values were greater than in the CEN 1811 sweat formulation. Of particular importance was that dissolution of tungsten in the full SSFL formulation with pH 6.5 was a factor of two higher than in the CEN 1811 formulation with the same pH but different chemical composition. The difference in dissolution was even more pronounced for cobalt powder. Dissolution of cobalt in the full SSFL formulation with pH 6.5 was a factor of four higher than in the CEN 1811 formulation with the same pH but different chemical composition. For the very poorly soluble tungsten carbide material, observed dissolution rates do not appear to be influenced by solvent pH or composition.

### EXAMPLE 10

### Characterization of Long-Term Stability of an Artificial Sebum Formulated to Closely Mimic Human Sebum

This example demonstrates a formulation of artificial sebum that can be used as a component of SSFL in studies for characterizing the release and subsequent partitioning and absorption of chemicals from materials in contact with skin.

Lipids and formulation of synthetic sebum. Cholesteryl oleate, oleoyl oleate, palmityl palmitate, cholesterol and vitamin E ((±)-α-tocopherol) were purchased from Sigma-Aldrich (St. Louis, Mo.). Squalene and stearic acid were from Acros Organics (Geel, Belgium). Triolein and tristearin were purchased from MP Biochemicals (Solon, Ohio), and oleic acid was from Fisher Scientific (Fairlawn, N.J.). The artificial sebum summarized in Table 3 was based on a formulation by Musial and Kubis (Polim. Med., 34: 17-30, 2004) that was modified to closely match median values of lipid constituents in human sebum and used more realistic proportions of saturated and monounsaturated components estimated from Stefaniak and Harvey (Toxicol. In Vitro 20: 1265-1283, 2006). All nine lipids were dissolved in a 2:1 co-solvent mixture of chloroform and methanol and divided into two portions, one of which received vitamin E at 0.1% by weight to mimic human sebum content. After combining and mixing, portions of lipid solution were transferred to 16×125 mm glass tubes equipped with Teflon-lined screw caps. One set of tubes with lipid solution was maintained at 4° C. to characterize storage using refrigeration. Solvent was removed from the remaining tubes by evaporation under nitrogen and the tubes with lipids split into two sets, one was maintained at 4° C. to characterize storage and the other at 32° C. to characterize use conditions (e.g., mimic human skin conditions). For each set, triplicate samples were harvested for analysis at 2, 4, 7, 9, 11, 14, 16, 18, 21, 23, 25, and 28 days.

Thin-layer chromatography. All samples were analyzed using thin layer chromatography. Glass plates (20×20 cm) coated with 0.25-mm-thick silica gel G (Adsorbosil-plus-1; Alltech Associates; Deerfield Ill.) were washed with chloroform:methanol, 2:1, activated in a 110° C. oven, and the adsorbent was scored into 6-mm-wide lanes. Calibrated glass capillaries were used to apply 5 μl samples two to three cm from the bottom edge of the plate, and the chromatogram was developed to 20 cm with hexane, followed by toluene to 20 cm, followed by hexane:ethyl ether:acetic acid, 70:30:1, to 12 cm. After development, chromatograms were air dried, sprayed with 50% sulfuric acid, and slowly heated to 220° C. on an aluminum slab on a hot plate. After two hrs, charring was complete, and the chromatograms were quantitated by photodensitometry. Thin layer chromatography analyses demonstrated that lipid reagents (a) squalene, (b) cholesteryl oleate, (c) cholesterol, (d) palmityl palmitate, (e) oleyl oleate, (f) tristearin, (g) triolein, (h) stearic acid, and (i) oleic acid) were pure materials. These analyses also indicated that under various conditions the individual lipids were chemically-stable (no cross-reactions) and that the formulation could be stored refrigerated for 28 days without loss of chemical integrity. For example, the sebum constituents maintained dry or in the chloroform:methanol solution at 4° C. were stable through 28 days in the presence and absence of vitamin E. In the presence of vitamin E, the artificial sebum formulation could be used for 28 days at a temperature similar to human skin. For example, all sebum lipids maintained dry at 32° C. were also stable in the presence of vitamin E; however, squalene oxidized completely by day 21 in the absence of vitamin E.

These studies demonstrate that the disclosed artificial formulation of sebum is representative of native human sebum (e.g., the artificial sebum formulation included all six lipid classes present in human sebum, the lipid content closely matched median values in human sebum, and the proportions of saturated and unsaturated wax esters and triglycerides closely matched human sebum).

In view of the many possible embodiments to which the principles of the disclosed invention may be applied, it should be recognized that the illustrated embodiments are only preferred examples of the invention and should not be taken as limiting the scope of the invention. Rather, the scope of the invention is defined by the following claims. We therefore claim as our invention all that comes within the scope and spirit of these claims.

We claim:

1. A composition comprising: electrolytes, inorganic constituents, organic acids, carbohydrates, nitrogenous substances and vitamins in concentrations found in human sweat, and maintained at a pH of 4.8-5.8.

2. The composition of claim 1, further comprising a sebum formulation, wherein the sebum formulation comprises squalene, wax esters, triglycerides, free fatty acids, cholesterol esters, free cholesterol and vitamin E in concentrations found in human sebum.

3. The composition of claim 1, wherein the electrolytes comprise bicarbonate, and the ionic constituents comprise sulfur, bromine, cadmium, copper, iodine, iron, lead, manganese, nickel and zinc.

4. The composition of claim 3, wherein the electrolytes comprise sodium, chloride, calcium, potassium, magnesium, phosphate and bicarbonate; the ionic constituents comprise sulfate, sulfur, fluoride, phosphorous, bromine, cadmium, copper, iodide, iron, lead, manganese, nickel, zinc; the organic acids comprise lactic acid, pyruvic acid, butyric acid, acetic acid, hexanoic acid, propionic acid, isobutyric acid, and isovaleric acid; the carbohydrates comprise glucose; the amino acids comprise alanine, arginine, aspartic acid, citrulline, glutamic acid, glycine, histidine, isoleucine, leucine, lysine, ornithine, phenylalanine, threonine, tyrosine, and valine; the nitrogenous substances comprise ammonium, uric acid, creatinine and creatine; and the vitamins comprise thiamine, riboflavin, nicotinic acid, pathothenic acid, pyridoxine, folic acid, ascorbic acid, dehydroascorbic acid, inositol, choline and p-aminobenzoic acid, at a pH of 5.1-5.5.

5. The composition of claim 4, wherein the electrolytes are present in a molar concentration of Na (3.0-3.5×10⁻²), Cl (2.0-2.5×10⁻²), Ca (5.0-5.5×10⁻³), K (5.8-6.2×10⁻³), Mg (8.0-8-5×10⁻⁵), PO₄ (2.8-3.4×10⁻⁴), and HCO₃ (2.7-3.3×10⁻³); the ionic constituents are present in a concentration of SO₄ (3.9-4.5×10⁻⁴), S (2.0-2.6×10⁻³), F (0.8-1.4×10⁻⁵), P (1.0-1.6×10⁻⁵), Br (2.0-2.6×10⁻⁶), Cd (1.5-2.1×10⁻⁸), Cu (9.1-9.7×10⁻⁷), I (6.8-7.4×10⁻⁸), Fe (9.5-10.1×10⁻⁶), Pb (0.9-1.5×10⁻⁷), Mn (0.8-1.4×10⁻⁶), Ni (3.9-4.5×10⁻⁷), and Zn (1.0-1.6×10⁻⁵); the organic acids and carbohydrates are present in concentrations of lactic acid (1.1-1.7×10⁻²), pyruvic acid (1.5-2.1×10⁻⁴), butyric acid (2.1-2.7×10⁻⁶), acetic acid (1.0-1.6×10⁻⁴), hexanoic acid (8.7-9.3×10⁻⁷), propionic acid (3.2-3.8×10⁻⁶), isobutyric acid (7.7-8.3×10⁻⁷), isovaleric acid (0.8-1.4×10⁻⁶), and glucose (1.4-2.0×10⁻⁴); the amino acids are present in concentrations of alanine (3.3-3.9×10⁻⁴), arginine (7.5-8.1×10⁻⁴), aspartic acid (3.1-3.7×10⁻⁴), citrulline (3.7-4.3×10⁻⁴), glutamic acid (3.4-4.0×10⁻⁴), glycine (3.6-4.2×10⁻⁴), histidine (4.9-5.5×10⁻⁴), isoleucine (1.4-2.0×10⁻⁴), leucine (1.8-2.4×10⁻⁴), lysine (1.2-1.8×10⁻⁴), ornithine (1.2-1.8×10⁻⁴), phenylalanine (1.0-1.6×10⁻⁴), threonine (4.2-4.8×10⁻⁴), tryptophan (5.2-5.8×10⁻⁵), tyrosine (1.4-2.0×10⁻⁴), and valine (2.2-2.8×10⁴); the nitrogenous substances are present in a concentration of NH₃ (4.9-5.5×10⁻³), urea (0.7-1.3×10⁻²), uric acid (5.6-6.2×10⁻⁵), creatinine (8.1-8.7×10⁻⁵), and creatine (1.2-1.8×10⁻⁵); the vitamins are present in a concentration of thiamine (4.7-5.3×10⁻³), riboflavin (1.7-2.5×10⁻³), niacin (3.8-4.4×10⁻¹), pantothenic acid (1.0-1.6×10⁻¹), pyridoxine (0.7-1.3×10⁻⁸), folic acid (1.3-1.9×10⁻⁸), ascorbic acid (0.7-1.3×10⁻⁵) and/or its oxidation product dehydroascorbic acid (0.8-1.4×10⁻⁵), inositol (1.3-1.9×10⁻⁶), choline (2.3-2.9×10⁻⁵), and p-aminobenzoic acid (6.8-7.4×10⁻⁸).

6. The composition of claim 5, wherein the electrolytes are present in concentrations of Na (3.1×10⁻²), Cl (2.3×10⁻²), Ca (5.2×10⁻³), K (6.1×10⁻³), Mg (8.2×10⁻⁵), PO₄ (3.1×10⁻⁴), and HCO₃ (3×10⁻³); ionic constituents are present in a concentration of SO₄ (4.2×10⁻⁴), S (2.3×10⁻³), F (1.1×10⁻⁵), P (1.3×10⁻⁵), Br (2.3×10⁻⁶), Cd (1.8×10⁻⁸), Cu (9.4×10⁻⁷), I (7.1×10⁻⁸), Fe (9.8×10⁻⁶), Pb (1.2×10⁻⁷), Mn (1.1×10⁻⁶), Ni (4.2×10⁻⁷), and Zn (1.3×10⁻⁵); organic acids and carbohydrates are present in concentrations of lactic acid (1.4×10⁻²), pyruvic acid (1.8×10⁻⁴), butyric acid (2.4×10⁻⁶), acetic acid (1.3×10⁻⁴), hexanoic acid (9.0×10⁻⁷), propionic acid (3.5×10⁻⁶), isobutyric acid (8.0×10⁻⁷), isovaleric acid (1.1×10⁻⁶), and glucose (1.7×10⁻⁴); amino acids are present in concentrations of alanine (3.6×10⁻⁴), arginine (7.8×10⁻⁴), aspartic acid (3.4×10⁻⁴), citrulline (4.0×10⁻⁴), glutamic acid (3.7×10⁻⁴), glycine (3.9×10⁻⁴), histidine (5.2×10⁻⁴), isoleucine (1.7×10⁻⁴), leucine (2.1×10⁻⁴), lysine (1.5×10⁻⁴), ornithine (1.5×10⁻⁴), phenylalanine (1.3×10⁻⁴), threonine (4.5×10⁻⁴), tryptophan (5.5×10⁻⁵), tyrosine (1.7×10⁻⁴), and valine (2.5×10⁻⁴); the nitrogenous substances are present in concentrations of NH₃ (5.2×10⁻³), urea (1.0×10⁻²), uric acid (5.9×10⁻⁵), creatinine (8.4×10⁻⁵), and creatine (1.5×10⁻⁵); the vitamins are present in concentrations of thiamine (5.0×10⁻³), riboflavin (2.0×10⁻³), niacin (4.1×10⁻¹), pantothenic acid (1.3×10⁻¹), pyridoxine (1.0×10⁻⁸), folic acid (1.6×10⁻⁸), ascorbic acid (1.0×10⁻⁵) and/or its oxidation product dehydroascorbic acid (1.1×10⁻⁵), inositol (1.6×10⁻⁶), choline (2.6×10⁻⁵), and p-aminobenzoic acid (7.1×10⁻⁸); and the composition is at a pH of about 5.3.

7. The composition of claim 2, wherein the sebum formulation comprises 8-12% squalene, 23-27% wax esters, 30-35% triglycerides, 25-30% free fatty acids, 1-3% cholesterol esters, and 3-5% free cholesterol.

8. The composition of claim 2, wherein the sebum formulation comprises 10% squalene, 25% wax esters, 33% triglycerides, 28% free fatty acids, 2% cholesterol esters, and 4% free cholesterol.

9. The composition of claim 7, wherein the wax esters comprise palmityl palmitate and oleyl oleate; the triglycerides comprise tristearin and triolein; the free fatty acids comprise stearic, palmitic and oleic acid; the cholesterol esters comprise cholesteryl oleate; and the Vitamin E comprises (±)-α-tocopherol.

10. The composition of claim 8, wherein the sebum formulation comprises:

| Constituent | Saturation | Chemical |
|---|---|---|
| Squalene | | Squalene |
| Wax esters | Saturated | Palmityl Palmitate |
| | Unsaturated | Oleyl Oleate |
| Triglycerides | Saturated | Tristearin |
| | Unsaturated | Triolein |
| Free Fatty Acids | Saturated | Stearic/Palmitic Acids |
| | Unsaturated | Oleic Acid |
| Cholesterol Esters | | Cholesteryl Oleate |
| Free Cholesterol | | Cholesterol |
| Vitamin E | | (±)-α-Tocopherol |

11. An artificial sweat composition comprising:

| Constituents | Mass (g/L) |
|---|---|
| Primary electrolytes and ionic constituents | |
| Sodium Sulfate (Na₂SO₄) | 5.83 × 10⁻² |
| Sodium Iodide (NaI) | 1.06 × 10⁻⁵ |
| Sodium Fluoride (NaF) | 4.62 × 10⁻⁴ |
| Sodium Bromide (NaBr) | 2.37 × 10⁻⁴ |
| Cadmium Chloride Anhydrous (CdCl₂) | 3.30 × 10⁻⁶ |
| Copper (II) Chloride Dihydrate (CuCl₂•2H₂O) | 1.60 × 10⁻⁴ |
| Ammonium Hydroxide (NH₄OH) | 1.82 × 10⁻¹ |
| Sulfur (S) | 7.37 × 10⁻² |
| Iron Sulfate Heptahydrate (FeSO₄•7H₂O) | 2.72 × 10⁻³ |
| Lead (Pb)-Reference Solution 1000 ppm | 2.49 × 10⁻⁵ |
| Manganese (II) Chloride (MnCl₂) | 1.38 × 10⁻⁴ |
| Nickel (Ni)-Reference Solution 1000 ppm | 2.46 × 10⁻⁵ |
| Zinc (Zn)-Reference Solution 1000 ppm | 8.50 × 10⁻⁴ |
| Sodium Bicarbonate (NaHCO₃) | 2.52 × 10⁻¹ |
| Potassium Chloride (KCl) | 4.55 × 10⁻¹ |
| Magnesium Chloride Hexahydrate (MgCl₂•6H₂O) | 1.67 × 10⁻² |
| Sodium Phosphate Anhydrous Monobasic (NaH₂PO₄) | 4.84 × 10⁻² |
| Calcium Chloride Dihydrate (CaCl₂•2H₂O) | 7.65 × 10⁻¹ |
| Phosphorous Pentachloride (PCl₅) | 2.71 × 10⁻³ |
| Sodium Chloride (NaCl)¹ | 5.84 × 10⁻² |
| Organic acids and carbohydrates | |
| Lactic acid (CH₃CH(OH)COONa) | 1.57 × 10⁰ |
| Pyruvic acid (C₃H₄O₃) | 1.59 × 10⁻² |
| Butyric acid (C₄H₈O₂) | 2.11 × 10⁻⁴ |
| Acetic acid (C₂H₄O₂) | 7.81 × 10⁻³ |
| Hexanoic acid (C₆H₁₂O₂) | 1.05 × 10⁻⁴ |
| Propionic acid (C₃H₆O₂) | 2.59 × 10⁻⁴ |
| Isobutyric acid (C₄H₈O₂) | 7.05 × 10⁻⁵ |
| Isovaleric acid (C₅H₁₀O₂) | 1.12 × 10⁻⁴ |
| D(+)-Glucose (C₆H₁₂O₆) | 3.06 × 10⁻² |
| Amino acids | |
| DL-Alanine (C₃H₇NO₂) | 5.11 × 10⁻² |
| L-(+)-Arginine (C₆H₁₄N₄O₂) | 1.36 × 10⁻¹ |
| L-(+)-Aspartic acid (C₄H₇NO₄) | 4.53 × 10⁻² |
| L-(+)-Citrulline (C₆H₁₃N₃O₃) | 7.01 × 10⁻² |
| L-(+)-Glutamic acid (C₅H₉NO₄) | 5.44 × 10⁻² |
| Glycine (C₂H₅NO₂) | 2.93 × 10⁻² |
| L-Histidine (C₆H₉N₃O₂) | 8.07 × 10⁻² |
| L-Isoleucine (C₆H₁₃NO₂) | 2.23 × 10⁻² |
| L-Leucine (C₆H₁₃NO₂) | 2.75 × 10⁻² |
| L-(+)-Lysine Monohydrochloride (C₆H₁₄N₂O₂•HCl) | 2.74 × 10⁻² |
| L-(+)-Ornithine Monohydrochloride (C₅H₁₂N₂O₂•HCl) | 2.53 × 10⁻² |
| L-Phenylalanine (C₉H₁₁NO₂) | 2.13 × 10⁻² |
| L-Threonine (C₄H₉NO₃) | 5.36 × 10⁻² |
| L-(−)-Tryptophan (C₁₁H₁₂N₂O₂) | 1.12 × 10⁻² |
| L-Tyrosine (C₉H₁₁NO₃) | 3.08 × 10⁻² |
| L-Valine (C₅H₁₁NO₂) | 2.93 × 10⁻² |
| Nitrogenous Substances | |
| Ammonium | — |
| Uric acid (C₅H₄N₄O₃) | 9.92 × 10⁻³ |
| Urea (CH₄N₂O) | 6.01 × 10⁻¹ |
| Creatinine (C₄H₇N₃O) | 9.50 × 10⁻³ |
| Creatine Monohydrate (C₄H₉N₃O₂•H₂O) | 2.24 × 10⁻³ |
| Vitamins | |
| Thiamine Hydrochloride (C₁₂H₁₇ClN₄OS•HCl) | 1.69 × 10⁰ |
| Riboflavin (C₁₇H₂₀N₄O₆) | 7.53 × 10⁻¹ |
| Nicotinic Acid (C₆H₅NO₂) | 5.05 × 10¹ |
| D-Pantothenic Acid (C₉H₁₇NO₅) | 2.85 × 10¹ |
| Pyridoxine Hydrochloride (C₈H₁₁NO₃•HCl) | 2.06 × 10⁻⁶ |
| Folic Acid (C₁₉H₁₉N₇O₆) | 7.06 × 10⁻⁶ |
| L-(+)-Ascorbic Acid (C₆H₈O₆) | 1.76 × 10⁻³ |
| Dehydroascorbic Acid | 1.91 × 10⁻³ |
| Inositol (C₆H₁₂O₆) | 2.88 × 10⁻⁴ |
| Choline Chloride (C₅H₁₄NOCl) | 3.63 × 10⁻³ |
| p-Aminobenzoic Acid (C₇H₇NO₂) | 9.73 × 10⁻⁶ |

12. A method of making the artificial sweat composition of claim 1, comprising:
providing a volume of water of about 70-80% of a final desired liquid volume of the artificial sweat composition;
first adding the primary electrolytes and ionic constituents to the water to form a primary solution;
then adding the organic acids, amino acids, nitrogenous substances and vitamins to the primary solution.

13. A method of storing the composition of claim 2, comprising placing it in a container under a nitrogen gas blanket at 4° C.

14. The method of claim 14, further comprising storing the composition at a pH greater than 5.

15. A method of testing dissolution of an object in human sweat, comprising
exposing the object to the composition of claim 1 for a sufficient period of time for it to at least partially dissolve;
quantifying dissolution of the object in the composition.

16. The method of claim 14, wherein the composition further comprises artificial sebum.

17. The method of claim 15, wherein the artificial sweat and sebum are present in a mass ratio of at least 1% artificial sebum.

18. The method of claim 15, wherein the artificial sebum is present in a layer present in the composition while exposing the object to the composition.
